(19) United States
(12) Patent Application Publication
(10) Pub. No.: US 2024/0418730 A1
(43) Pub. Date: Dec. 19, 2024

(54) HOST CELL PROTEIN ANALYSIS FOR ADENO-ASSOCIATED VIRUS (AAV)-BASED GENE THERAPY

(71) Applicant: Regeneron Pharmaceuticals, Inc., Tarrytown, NY (US)

(72) Inventors: Haibo Qiu, Millwood, NY (US); Yunli Hu, Tarrytown, NY (US); Mengqi Hu, White Plains, NY (US); Ning Li, New Canaan, CT (US)

(21) Appl. No.: 18/745,346

(22) Filed: Jun. 17, 2024

Related U.S. Application Data
(60) Provisional application No. 63/521,780, filed on Jun. 19, 2023.
(60) Provisional application No. 63/659,090, filed on Jun. 12, 2024.

(51) Int. Cl. G01N 33/68; G01N 33/569

(52) U.S. Cl. CPC G01N 33/6848; G01N 33/56983; G01N 2333/015; G01N 2333/976

(57) ABSTRACT

The present invention provides methods for identifying, quantifying, and/or characterizing at least one host cell protein (HCP) impurity in a sample containing AAV vectors. The HCP impurities can be enriched through a differential digestion, maintaining intact capsids while exposing the sample to mild denaturation. The mildly denatured sample can subsequently be subjected to enzymatic digestion, generating peptides which can be identified and quantified by liquid chromatography-mass spectrometry (LC-MS) analysis to identify, quantify and/or characterize said at least one HCP impurity.

### CROSS-REFERENCE TO RELATED APPLICATIONS

This application claims priority to and the benefit of U.S. Provisional Application No. 63/521,780, filed Jun. 19, 2023 and also claims priority to and the benefit of U.S. Provisional Application No. 63/659,090, filed Jun. 12, 2024 each of which is incorporated herein by reference in its entirety for all purposes.

### BACKGROUND

Gene therapy biopharmaceuticals mediate therapeutic effects by transcription and/or translation of transferred genetic material, such as integrating genetic material into the host genome, and are used to treat, prevent or cure a disease. Currently, gene therapy is one of the most investigated therapeutic modalities in preclinical and clinical settings. However, gene therapy experienced a major setback in the late 1990's and early 2000's which raised concerns about the safety of gene therapy and highlighted the critical need for safer gene delivery vectors. A better understanding of gene delivery vectors and advancing the manufacture of safe and effective vectors is necessary to mitigate safety risks.

Gene delivery vectors are essential to ensure efficient gene delivery to the target tissue and cells. The ideal gene delivery system should have high gene transfer efficiency, low toxicity to the cell, and single cell specificity to the intended target. Based on gene delivery vector types, vectors can be divided into non-viral vectors and viral vectors. Due to the high gene transfer efficiency of viral vectors, they have been widely used in clinical trials.

Adeno-associated virus (AAV) is the most widely used viral vector for in vivo gene therapy applications. AAVs have low immunogenicity and can enable long-term, stable gene expression. The use of AAVs for gene therapy has created the need for analytical methods to monitor and characterize these products. Process-related and product-related impurities should be monitored to ensure product quality and process consistency.

Biopharmaceutical products must meet very high standards of purity. Thus, it is important to monitor any impurities in such biopharmaceutical products at different stages of drug development, production, storage and handling. Residual impurities should be at an acceptable low level prior to conducting clinical studies. Residual impurities are also a concern for biopharmaceutical products intended for end-users. For example, host cell proteins (HCPs) can be present in biopharmaceuticals which are developed using cell-based systems. The presence of HCPs in drug products should be monitored and can be unacceptable above a certain threshold, depending on the product and the particular HCP. Sometimes, even trace amounts of HCPs can cause an immunogenic response in an end-user.

It will be appreciated that a need exists for methods to identify and quantitate HCPs to monitor and control the residual HCPs in a drug substance or other product to mitigate safety risks.

### SUMMARY

This disclosure provides methods of identifying, quantifying and/or characterizing HCP impurities in a sample. In some exemplary embodiments, the methods can comprise: (a) contacting a sample containing at least one HCP impurity and at least one AAV vector to a reducing agent to produce a reduced sample; (b) subjecting said reduced sample to mild denaturation conditions to produce a partially denatured sample; (c) subjecting said partially denatured sample to enzymatic digestion conditions to produce a peptide digest; and (d) subjecting said peptide digest to liquid chromatography-mass spectrometry (LC-MS) analysis to identify, quantify and/or characterize said at least one HCP impurity.

In one aspect, the reducing agent is selected from TCEP or DTT. In a specific aspect, the reducing agent is TCEP.

In one aspect, the mild denaturation conditions comprise a temperature from about 35° C. to about 65° C. In a specific aspect, the temperature is about 35° C., about 40° C., about 45° C., about 50° C., about 55° C., about 65° C., about 70° C., or about 75° C.

In one aspect, the enzymatic digestion conditions comprise contacting the partially denatured sample to at least one digestive enzyme. In a specific aspect, the at least one digestive enzyme comprises trypsin.

In one aspect, the enzymatic digestion conditions include trypsin at an enzyme to substrate ratio from about 1:10 to about 1:4000. In a specific aspect, the enzymatic digestion conditions include trypsin at an enzyme to substrate ratio of about 1:20, about 1:400, or about 1:2000. In another specific aspect, the enzymatic digestion conditions include trypsin at an enzyme to substrate ratio of about 1:20.

In one aspect, the duration of the enzymatic digestion conditions is from 1 to 20 hours. In another specific aspect, the duration of the enzymatic digestion conditions is about 2 hours, about 4 hours, or about 18 hours.

In one aspect, the amount of the peptide digest injected on LC-MS is from about 1 μg to about 4 μg, about 1 μg, about 1.5 μg, about 2 μg, about 3 μg, or about 4 μg.

In one aspect, the method further comprises enriching said peptide digest prior to step (d). In a specific aspect, the method further comprises centrifuging said peptide digest to separate peptides from undigested or partially digested proteins or AAV vectors.

This disclosure also provides methods of identifying, quantifying and/or characterizing at least one host cell protein (HCP) impurity in the capsid of an AAV vector. In some exemplary embodiments, the methods can comprise: (a) separating a sample including at least one HCP impurity in the capsid of an AAV vector from free HCPs to produce an enriched AAV vector sample; (b) treating the enriched AAV vector sample to a reducing agent to produce a reduced sample; (c) subjecting the reduced sample to denaturation conditions to produce a denatured sample; (d) treating the denatured sample to enzymatic digestion conditions to produce a peptide digest; and (e) subjecting the peptide digest to liquid chromatography-mass spectrometry (LC-MS) analysis to identify, quantify and/or characterize said at least one HCP impurity.

In one aspect, the reducing agent is selected from TCEP or DTT. In a specific aspect, the reducing agent is DTT.

In one aspect, the enzymatic digestion conditions comprise contacting the denatured sample to at least one digestive enzyme. In a specific aspect, the at least one digestive enzyme comprises trypsin.

In one aspect, a duration of the enzymatic digestion conditions is from 1 to 20 hours, about 2 hours, about 4 hours, or about 18 hours. In a specific aspect, the duration is about 4 hours.

In one aspect, the amount of the peptide digest injected on LC-MS is from about 1 μg to about 4 μg, about 1 μg, about 1.5 μg, about 2 μg, about 3 μg, or about 4 μg.

This disclosure provides additional methods of enriching and identifying at least one host cell protein (HCP) impurity in a sample. In some exemplary embodiments, the methods can comprise treating the sample with a reducing agent, followed by a denaturation step, followed by enzymatic digestion, and identification of said at least one enriched HCP using LC-MS.

In one aspect, the sample includes a supernatant and a pellet.

In one aspect, the reducing agent is selected from TCEP or DTT.

In one aspect, the denaturation of the sample is performed at a temperature from about 35° C. to about 80° C. In a specific aspect, the temperature is about 35° C., about 40° C., about 45° C., about 50° C., about 55° C., about 65° C., about 70° C. or about 75° C.

In one aspect, the enzymatic digestion conditions comprise contacting the denatured sample to at least one digestive enzyme. In a specific aspect, the at least one digestive enzyme comprises trypsin.

In one aspect, the enzymatic digestion conditions include trypsin at an enzyme to substrate ratio from about 1:10 to about 1:4000. In a specific aspect, the enzymatic digestion conditions include trypsin at an enzyme to substrate ratio of about 1:20, about 1:400, or about 1:2000. In another specific aspect, the enzymatic digestion conditions include trypsin at an enzyme to substrate ratio of about 1:20.

In one aspect, the duration of the enzymatic digestion conditions is from 1 to 20 hours. In another specific aspect, the duration of the enzymatic digestion conditions is about 2 hours, about 4 hours or about 18 hours. In another specific aspect, the duration of the enzymatic digestion condition is about 4 hours.

In one aspect, the amount of the peptide digest injected on LC-MS ranges from about 1 μg to 2 μg.

This disclosure further provides methods for identifying, quantifying, and/or characterizing at least one non-viral protein in a sample including at least one virus or viral vector. In some exemplary embodiments, the methods can comprise: (a) subjecting a sample including at least one non-viral protein and at least one virus or viral vector to partially denaturing conditions to form a partially denatured sample, wherein said partially denaturing conditions are capable of substantially denaturing said at least one non-viral protein and do not substantially denature said at least one virus or viral vector; (b) subjecting said partially denatured sample to digestion conditions to form a peptide digest; and (c) subjecting said peptide digest to LC-MS analysis to identify, quantify, and/or characterize said at least one non-viral protein.

In one aspect, the at least one non-viral protein is a host cell protein.

In one aspect, the at least one virus or viral vector is an AAV vector. In a specific aspect, the AAV comprises a serotype selected from a group consisting of AAV1, AAV2, AAV3, AAV4, AAV5, AAV6, AAV7, AAV8, AAVrh8, AAV2/8, AAV9, AAV10, AAV11, AAV12, variations thereof and combinations thereof.

In one aspect, the viral vector is a therapeutic vector.

In one aspect, the method further comprises subjecting the sample to a reducing step prior to or concurrent with step (a). In a specific aspect, the reducing step comprises contacting the sample to TCEP or DTT.

In one aspect, said partially denaturing conditions comprise a temperature from about 35° C. to about 65° C. In a specific aspect, said temperature is about 35° C., about 40° C., about 45° C., about 50° C., about 55° C., about 65° C., about 70° C. or about 75° C.

In one aspect, said digestion conditions comprise contacting said partially denatured sample to at least one digestive enzyme, optionally wherein said at least one digestive enzyme comprises trypsin. In a specific aspect, said trypsin is present at an enzyme to substrate ratio of from 1:10 to 1:4000, about 1:20, about 1:400, or about 1:2000.

In one aspect, a duration of said digestion conditions is from 1 to 20 hours, about 2 hours, for about 4 hours or for about 18 hours.

In one aspect, an amount of said peptide digest injected on LC-MS is from about 1 μg to about 4 μg, about 1 μg, about 1.5 μg, about 2 μg, about 3 μg, or about 4 μg.

In one aspect, the method further comprises quantifying a ratio of the abundance of the at least one non-viral protein to an abundance of a protein of said at least one virus or viral vector. In a specific aspect, said ratio is at least 0.5, from about 0.5 to 10, about 0.5, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10.

In one aspect, the host cell protein is a Chinese hamster ovary (CHO) protein or a human protein.

In one aspect, the liquid chromatography system is selected from a group consisting of reversed phase chromatography, ion exchange chromatography, size exclusion chromatography, affinity chromatography, hydrophobic interaction chromatography, hydrophilic interaction chromatography, mixed-mode chromatography, or a combination thereof.

In one aspect, the mass spectrometry system is an electrospray ionization mass spectrometer, nano-electrospray ionization mass spectrometer, or an Orbitrap-based mass spectrometer, wherein said mass spectrometer is coupled to said liquid chromatography system.

In one aspect, the method further comprises enriching said peptide digest prior to step (c). In a specific aspect, the method further comprises centrifuging said peptide digest to separate peptides from undigested or partially digested proteins, viruses or viral vectors.

This disclosure additionally provides methods for characterizing at least one HCP in a sample including a virus or viral vector. In some exemplary embodiments, the methods can comprise: (a) quantifying and/or characterizing an HCP outside of a virus or viral vector in a sample including at least one HCP and at least one virus or viral vector by: (i) subjecting the sample to partially denaturing conditions to form a partially denatured sample, wherein said partially denaturing conditions are capable of substantially denaturing said at least one HCP and do not substantially denature said at least one virus or viral vector; (ii) subjecting said partially denatured sample to digestion conditions to form a peptide digest; and (iii) subjecting said peptide digest to LC-MS analysis to quantify and/or characterize the HCP outside of the virus or viral vector; (b) quantifying and/or characterizing said HCP inside of said virus or viral vector by: (i) separating said at least one virus or viral vector from free HCPs in said sample to produce an enriched sample; (ii) treating said enriched sample to a reducing agent to produce a reduced sample; (iii) subjecting said reduced sample to denaturing conditions to produce a denatured sample; (iv) subjecting said denatured sample to enzymatic digestion conditions to produce a peptide digest; and (v) subjecting said peptide digest to LC-MS analysis to quantify and/or characterize the HCP inside of the virus or viral vector; and (c) comparing the quantification and/or characterization of step (a) to the quantification and/or characterization of step (b) to characterize said at least one HCP.

This disclosure also provides methods for enriching a virus or viral vector. In some exemplary embodiments, the methods can comprise: (a) subjecting a sample including a virus or viral vector and at least one non-viral protein to partially denaturing conditions to produce a partially denatured sample, wherein said partially denatured conditions are capable of substantially denaturing said at least one non-viral protein and do not substantially denature said virus or viral vector; (b) subjecting said partially denatured sample to enzymatic digestion conditions to form a partially digested sample, wherein said enzymatic digestion conditions are capable of substantially digesting denatured proteins and do not substantially denature native proteins; and (c) subjecting said partially digested sample to a separation step to enrich said virus or viral vector.

In one aspect, said at least one non-viral protein is a host cell protein. In a specific aspect, said host cell protein is a Chinese hamster ovary (CHO) protein or a human protein.

In one aspect, said at least one virus or viral vector is an AAV vector. In a specific aspect, said AAV vector comprises a serotype selected from a group consisting of AAV1, AAV2, AAV3, AAV4, AAV5, AAV6, AAV7, AAV8, AAVrh8, AAV2/8, AAV9, AAV10, AAV11, AAV12, variations thereof and combinations thereof.

In one aspect, said viral vector is a therapeutic vector.

In one aspect, the method further comprises subjecting said sample to a reducing step prior to or concurrent with step (a). In a specific aspect, said reducing step comprises contacting said sample to TCEP or DTT.

In one aspect, said partially denaturing conditions comprise a temperature from about 35° C. to about 65° C. In a specific aspect, said temperature is about 35° C., about 40° C., about 45° C., about 50° C., about 55° C., about 65° C., about 70° C. or about 75° C.

In one aspect, said digestion conditions comprise contacting said partially denatured sample to at least one digestive enzyme, optionally wherein said at least one digestive enzyme comprises trypsin. In a specific aspect, said trypsin is present at an enzyme to substrate ratio of from 1:10 to 1:4000, about 1:20, about 1:400, or about 1:2000.

In one aspect, a duration of said digestion conditions is from 1 to 20 hours, about 2 hours, for about 4 hours or for about 18 hours.

In one aspect, the separation step comprises centrifugation.

This disclosure additionally provides methods for enriching at least one fragment of at least one non-viral protein in a sample including at least one virus or viral vector. In some exemplary embodiments, the methods can comprise: (a) subjecting a sample including at least one non-viral protein and at least one virus or viral vector to partially denaturing conditions to produce a partially denatured sample; (b) subjecting said partially denatured sample to digestion conditions to produce a peptide digest; and (c) subjecting said peptide digest to a separation step to enrich said at least one fragment of said at least one non-viral protein.

In one aspect, said at least one non-viral protein is a host cell protein. In a specific aspect, said host cell protein is a Chinese hamster ovary (CHO) protein or a human protein.

In one aspect, said at least one virus or viral vector is an AAV vector. In a specific aspect, said AAV vector comprises a serotype selected from a group consisting of AAV1, AAV2, AAV3, AAV4, AAV5, AAV6, AAV7, AAV8, AAVrh8, AAV2/8, AAV9, AAV10, AAV11, AAV12, variations thereof and combinations thereof.

In one aspect, said viral vector is a therapeutic vector.

In one aspect, the method further comprises subjecting said sample to a reducing step prior to or concurrent with step (a). In a specific aspect, said reducing step comprises contacting said sample to TCEP or DTT.

In one aspect, said partially denaturing conditions comprise a temperature from about 35° C. to about 65° C. In a specific aspect, said temperature is about 35° C., about 40° C., about 45° C., about 50° C., about 55° C., about 65° C., about 70° C. or about 75° C.

In one aspect, said digestion conditions comprise contacting said partially denatured sample to at least one digestive enzyme, optionally wherein said at least one digestive enzyme comprises trypsin. In a specific aspect, said trypsin is present at an enzyme to substrate ratio of from 1:10 to 1:4000, about 1:20, about 1:400, or about 1:2000.

In one aspect, a duration of said digestion conditions is from 1 to 20 hours, about 2 hours, for about 4 hours or for about 18 hours.

In one aspect, the separation step comprises centrifugation.

These, and other, aspects of the present invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the present invention.

### BRIEF DESCRIPTION OF THE DRAWINGS

FIG. 1 illustrates potential methods of enriching and identifying HCPs in an AAV vector sample, according to an exemplary embodiment.

FIG. 2 illustrates a novel differential digestion method of enriching and identifying HCPs in an AAV vector sample, according to an exemplary embodiment.

FIG. 3A shows a comparison of viral protein abundance after direct digestion and after differential digestion, according to an exemplary embodiment.

FIG. 3B illustrates AAV1 VP3 capsid cryoEM structure mapped with high abundance peptides detected by either differential digestion or direct digestion, according to an exemplary embodiment.

FIG. 4 shows mass photometry of an AAV vector after differential digestion, after direct digestion and a control (without treatment), according to an exemplary embodiment.

FIG. 5A shows the number of HCPs identified by the differential digestion method of the present invention compared to conventional direct digestion, according to an exemplary embodiment.

FIG. 5B shows the intensity difference of HCP peptides detected by the differential digestion method of the present invention compared to conventional direct digestion, according to an exemplary embodiment.

FIG. 6A shows various conditions of the present invention as well as the resultant number of HCPs identified and HCP to viral particle protein ratio, according to an exemplary embodiment.

FIG. 6B shows the number HCPs identified for each AAV vector sample analyzed using the conditions described in FIG. 6A, according to an exemplary embodiment.

FIG. 6C shows the ratio of HCPs to viral proteins detected for each AAV vector sample analyzed using the conditions described in FIG. 6A, according to an exemplary embodiment.

FIG. 7 shows a model of the effect of differential digestion parameters on HCP analysis that may be used to determine the optimal conditions for the differential digestion based on specific desirability entered, according to an exemplary embodiment.

FIG. 8A illustrates the consistency in HCPs identified using the differential digestion method of the present invention for three preparations, according to an exemplary embodiment.

FIG. 8B shows the repeatability of HCP abundance determinations for the three preparations as well as the average of the three preparations using the differential digestion method of the present invention, according to an exemplary embodiment.

FIG. 9A shows the number of HCPs identified for three different AAVs, comparing the differential digestion method of the present invention with two different injection amounts to conventional direct digestion, according to an exemplary embodiment.

FIG. 9B shows a comparison of extracted ion chromatograms for variable injection amounts of differentially digested AAV vector samples, according to an exemplary embodiment.

FIG. 10 shows the most abundant HCPs identified in AAV vector samples using direct digestion, according to an exemplary embodiment.

FIG. 11 shows the most abundant HCPs identified in AAV vector samples using differential digestion, according to an exemplary embodiment.

FIG. 12 shows potential problematic HCPs identified after differential digestion, according to an exemplary embodiment.

FIG. 13 illustrates a method for analysis of HCPs encapsulated in the capsid of AAV vectors, according to an exemplary embodiment.

FIG. 14A illustrates a comparison of HCPs identified by differential digestion, direct digestion, and digestion of the pellet following differential digestion, according to an exemplary embodiment.

FIG. 14B illustrates a comparison of the molecular weight and pI distribution of HCPs identified by differential digestion and not identified by differential digestion, according to an exemplary embodiment.

FIG. 15 shows the comparison of the abundance of two AAV peptides generated by both direct digestion and differential digestion, according to an exemplary embodiment.

FIG. 16 shows the number of HCPs identified at different enzyme to substrate ratios, according to an exemplary embodiment.

FIG. 17 shows the number of HCPs identified at different denaturation temperatures and incubation times, according to an exemplary embodiment.

FIG. 18 shows the repeatability of HCPs identified in three AAV5 preparations, according to an exemplary embodiment.

FIG. 19 shows the most abundant HCPs identified and quantified in AAV5 samples from three preparations, according to an exemplary embodiment.

FIG. 20 shows HCPs identified in the digestion of the pellet of an AAV5 sample, according to an exemplary embodiment.

FIG. 21 shows mass photometry measurements of AAV2 (a) without treatment and (b) after differential digestion, according to an exemplary embodiment.

FIG. 22 shows mass photometry measurements of AAV5 (a) without treatment and (b) after differential digestion, according to an exemplary embodiment.

FIG. 23 shows mass photometry measurements of AAV6 (a) without treatment and (b) after differential digestion, according to an exemplary embodiment.

FIG. 24 shows mass photometry measurements of AAV9 (a) without treatment and (b) after differential digestion, according to an exemplary embodiment.

FIG. 25A shows the number of HCPs identified in five different AAV serotypes, according to an exemplary embodiment.

FIG. 25B shows the distribution of HCPs identified in five different AAV serotypes, according to an exemplary embodiment.

FIG. 26 shows the most abundant HCPs identified and quantified in three preparations of an AAV5 sample, according to an exemplary embodiment.

### DETAILED DESCRIPTION

Adeno-associated viruses (AAVs) have been widely used as gene delivery vectors to deliver genetic material, such as delivering nucleic acids for gene therapy. AAVs provide the advantages of non-pathogenicity and low immunogenicity. AAVs are nonpathogenic members of the Parvoviridae family under Dependovirus genus and require helpers, such as Adenovirus or Herpesvirus, for infection (Venkatakrishnan et al., Structure and Dynamics of Adeno-Associated Virus Serotype 1 VP1-Unique N-Terminal Domain and Its Role in Capsid Trafficking, Journal of Virology, May 2013, vol. 87, no. 9, pages 4974-4984). AAV encapsulates a single-stranded DNA genome of about 4.8 kilobases (kb) in an icosahedral capsid which is made of a shell of capsid viral proteins. Recombinant AAV genomes are nonpathogenic and do not integrate into a host's genome, but exist as stable episomes that provide long-term expression. AAV serotypes make a very useful system for preferentially transducing specific cell types.

Overall, AAV-based therapy has the advantages of being non-pathogenic and non-toxic, having cell type-specific infection, and offering different serotypes with varying cell transduction efficiencies. A disadvantage is that AAV production, purification, and characterization are more complex compared to, for example, antibody therapies. Fully packaged AAVs consist of an icosahedral capsid containing an about 4.8 kb single-stranded genome. An empty capsid has a molecular weight of about 3750 kDa, while a full capsid with an about 4.7 kb single-stranded genome has a molecular weight of about 5100 kDa. The purity of AAVs is defined by several product-related impurities, including empty capsids, capsids containing partial or incorrect genomes, and aggregated or degraded capsid, as well as residual host cell proteins (HCP).

Recent advancements in AAV clinical activity have created a demand for large-scale AAV production. The manufacturing processes of AAV consist of upstream production of AAV vectors and downstream purification to remove impurities. Host cell proteins (HCPs) are a major class of process-related impurities and are considered a critical quality attribute. Enzyme-linked immunosorbent assay (ELISA) is commonly conducted as an AAV HCP release assay to monitor residual HCPs and ensure product purity.

In order to manufacture biopharmaceutical products, it is important that such products have high purity, since residual HCPs can compromise product safety and stability. The characterization of HCPs in viral vector production has posed many challenges. For example, the location of the HCPs must be considered. HCPs may be located inside the vector, associating with the target genome, and/or part of the capsid of the AAV. In addition, the AAV serotype may have a profound effect on the HCPs identified.

In order to identify HCPs, several approaches have been employed, such as gel electrophoresis and/or digestion coupled with liquid chromatography-mass spectrometry (LC-MS), or digestion followed by enrichment with liquid chromatography (LC) followed by LC-MS. The use of LC-MS for HCP profiling offers an alternative analytical approach to ELISA, thus mitigating the risk associated with antibody coverage. LC-MS allows for the identification of individual HCPs and facilitates monitoring of low abundance HCPs. A major challenge of HCP profiling by mass spectrometry is that the dynamic range between low abundance HCP and the drug substance is beyond the range of most current mass spectrometry. For therapeutic protein HCP analysis, multiple methods have been developed to overcome the dynamic range issues. The residual HCPs in therapeutic proteins are enriched through molecular weight cutoff filtration, polyclonal antibodies capture, or removal of the therapeutic protein with affinity purification using Protein A or Protein G. Recently, the native digestion method has gained popularity in monoclonal antibody (mAb) HCP analysis, because of its high sensitivity and simple workflow. However, these profiling methods for therapeutic protein HCPs are not readily applicable to the monitoring of HCPs in AAV. Most highly sensitive methods require several or even hundreds of milligrams of therapeutic protein for sample preparation. Such an amount is not feasible for AAV, because of the limited quantities of available samples. Moreover, AAV exhibits distinct characteristics compared to therapeutic proteins, thereby posing a hurdle to the direct application of mAb analysis methods to AAV. To date, AAV drug substance HCP analysis by LC-MS has been accomplished through direct digestion, gel-electrophoresis fractionation and SP3 methods. These methods either require extensive HCP fractionation steps or lack the sensitivity to detect low abundance HCPs. Therefore, the need for a simple and sensitive AAV HCP LC-MS profiling method persists.

In addition, the use of direct digestion, immunoprecipitation, native digestion and molecular weight cut off filtration techniques have been implemented. However, the dynamic concentration and location of HCPs in the AAVs and the limitations of sample size and AAV concentration are major challenges to monitor and remove HCP impurities. In order to overcome this issue, methods of quantifying, characterizing and identifying HCPs have been developed.

The present application provides methods to enrich HCPs using a differential digestion. In some exemplary embodiments, the differential digestion substantially leaves the AAV intact due to lower incubation temperature while denaturing and digesting HCPs, allowing for enrichment of HCPs.

This disclosure provides methods to satisfy the aforementioned demands by providing methods to identify HCPs in a biopharmaceutical product to mitigate safety risks. Exemplary embodiments disclosed herein satisfy the aforementioned demands and the long-felt needs.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule.

As used herein, the term “therapeutic protein” includes any of proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies.

In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., 28 BIOTECHNOLOGY AND GENETIC ENGINEERING REVIEWS 147-176 (2012), the entirety of which is herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” (bsAbs) includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or KA-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific are also contemplated.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

As used herein, a “protein pharmaceutical product” or “biopharmaceutical product” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

As used herein, a “sample” refers to a mixture of molecules that comprises at least a viral particle, such as an AAV particle, or an empty viral capsid, that is subjected to manipulation in accordance with the methods of the invention, including, for example, separating, analyzing, extracting, concentrating, profiling and the like.

As used herein, the term “impurity” can include any undesirable protein present in a protein sample or protein biopharmaceutical product. Impurities can include process and product-related impurities. The impurity can further be of known structure, partially characterized, or unidentified. Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

As used herein, the term “host-cell protein” (HCP) includes protein derived from a host cell. Host-cell proteins can be a process-related impurity which can be derived from the manufacturing process. In some exemplary embodiments, the types of HCP process-related impurities in the composition can be at least two.

The presence of a host cell protein in a biotherapeutic product may be considered to be a higher or lower risk based on a number of measurable factors. One such factor is the concentration or abundance (quantity) of an HCP impurity in a biotherapeutic product. An HCP may have no discernible impact at a low enough abundance, as measured by, for example, ELISA or mass spectrometry. The level at which an HCP may present a considerable risk, which may be considered an unacceptable level in a product and may be monitored as a critical quality attribute (CQA), may depend on the specific identity of the HCP. Particular HCPs may be known to present a risk at a particular level, for example depending on the level of enzymatic activity of an HCP that is an enzyme.

Relatedly, the criticality of the presence of an HCP may depend on the function of that HCP, in particular in relation to the components of the biotherapeutic product. For example, an HCP lipase that may or is known to degrade polysorbate that is present in the biotherapeutic product of interest may be closely monitored and may have a low threshold for how much of the HCP impurity can be allowed in the biotherapeutic product. Other HCPs of particular concern may be, for example, proteases that may or are known to degrade a protein of interest in the biotherapeutic product, or immunogenic HCPs that may or are known to cause an immune reaction when administered to a subject. Using the method of the present invention, a person skilled in the art may evaluate the abundance, distribution, and/or identity of an HCP impurity in the context of the biotherapeutic product of interest to determine if the HCP impurity is an HCP impurity of concern, and based on that determination may use chromatographic or other separation methods to remove the impurity when producing the biotherapeutic product.

The terms “peptide,” “protein” and “polypeptide” refer, interchangeably, to a polymer of amino acids and/or amino acid analogs that are joined by peptide bonds or peptide bond mimetics. The twenty naturally-occurring amino acids and their single-letter and three-letter designations are as follows: Alanine A Ala; Cysteine C Cys; Aspartic Acid D Asp; Glutamic acid E Glu; Phenylalanine F Phe; Glycine G Gly; Histidine H His; Isoleucine I He; Lysine K Lys; Leucine L Leu; Methionine M Met; Asparagine N Asn; Proline P Pro; Glutamine Q Gln; Arginine R Arg; Serine S Ser; Threonine T Thr; Valine V Val; Tryptophan w Trp; and Tyrosine Y Tyr.

A “vector,” as used herein, refers to a recombinant plasmid or virus that comprises a nucleic acid to be delivered into a host cell, either in vitro or in vivo.

A “recombinant viral vector” refers to a recombinant polynucleotide vector including one or more heterologous sequences (i.e., nucleic acid sequence not of viral origin).

A “recombinant AAV vector (rAAV vector)” refers to a polynucleotide vector including one or more heterologous sequences (i.e., nucleic acid sequence not of AAV origin) that may be flanked by at least one, e.g., two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins).

A “capsid” is the protein shell of a virus, which encloses the genetic material. Three viral capsid proteins, VP1, VP1 and VP3, form the viral icosahedral capsid of 60 subunits in a ratio of 1:1:10. A full capsid contains genetic material and is required to provide therapeutic benefit. An empty capsid lacks the genome and therefore lacks the ability to provide therapeutic benefit to the patient.

A “viral particle” refers to a particle composed of at least one viral capsid protein and an encapsulated viral genome. While AAV is described in this disclosure as a model virus or viral particle, it is contemplated that the disclosed methods can be applied to profile a variety of viruses, e.g., the viral families, subfamilies, and genera. In some aspects, the viral capsid, virus, or viral particle belongs to a viral family selected from the group consisting of Adenoviridae, Parvoviridae, Retroviridae, Baculoviridae, and Herpesviridae. In some aspects, the viral capsid, virus, or viral particle belongs to a viral genus selected from the group consisting of Atadenovirus, Aviadenovirus, Ichtadenovirus, Mastadenovirus, Siadenovirus, Ambidensovirus, Brevidensovirus, Hepandensovirus, Iteradensovirus, Penstyldensovirus, Amdoparvovirus, Aveparvovirus, Bocaparvovirus, Copiparvovirus, Dependoparvovirus, Erythroparvovirus, Protoparvovirus, Tetraparvovirus, Alpharetrovirus, Betaretrovirus, Deltaretrovirus, Epsilonretrovirus, Gammaretrovirus, Lentivirus, Spumavirus, Alphabaculovirus, Betabaculovirus, Deltabaculovirus, Gammabaculovirus, Iltovirus, Mardivirus, Simplexvirus, Varicellovirus, Cytomegalovirus, Muromegalovirus, Proboscivirus, Roscolovirus, Lymphocryptovirus, Macavirus, Percavirus, and Rhadinovirus.

As used herein, the term “vector” refers to a recombinant plasmid or virus (“viral vector”) that comprises a nucleic acid to be delivered into a host cell, either in vitro or in vivo. Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, for example, interferon-mediated responses; (iii) wild type AAVs have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected).

In some exemplary embodiments, the sample can be prepared prior to LC-MS analysis. Preparation steps can include reduction, denaturation, alkylation, dilution, digestion, and separation (for example, centrifugation).

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

Proteins may have distinctive susceptibility to denaturation. For example, viral proteins assembled into a viral capsid may have reduced susceptibility to denaturation compared to a monomeric or smaller multimeric protein, for example a host cell protein. A difference in susceptibility to denaturation may be taken advantage of in order to preferentially denature a particular protein or class of proteins while leaving another protein or class of proteins in a substantially natively folded state. In some aspects, denaturation may be performed at a temperature selected to substantially denature a protein or class of proteins, for example non-viral proteins such as host cell proteins, while leaving the proteins of a viral capsid substantially in a folded state. This may be referred to as, for example, mild denaturation, limited denaturation, partial denaturation, or differential denaturation. A partially denatured sample may then be subjected to a digestion step to produce a peptide digest. Because denatured proteins are more susceptible to digestion by digestive enzymes, the peptide digest will preferentially include peptides from the more denatured proteins, for example non-viral proteins such as HCPs, compared to peptides from the more natively folded proteins, for example viral capsid proteins. This may be referred to as, for example, partial digestion, differential digestion, or limited digestion. The peptide digest will be enriched for peptides of the non-viral protein, for example HCPs, relative to the original sample. This enrichment may be useful for subsequent analysis, for example liquid chromatography-mass spectrometry analysis, in order to sensitively and accurately identify, characterize, and quantify the non-viral proteins.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, 12 JOURNAL OF PROTEOME RESEARCH 1067-1077 (2013)).

Conventional methods use a digestive enzyme in conditions and concentrations sufficient to completely digest all protein in a sample prior to LC-MS analysis. The present disclosure surprisingly finds that identification and quantification of low-abundance proteins such as HCPs can be improved through limited digestion, meaning that denaturation and digestion conditions are selected such that proteins in a sample are not completely digested. In some exemplary embodiments, proteins are subjected to mild denaturation or partial denaturation prior to digestion, such that a particular protein or class of proteins that are more susceptible to denaturation are preferentially digested.

In some aspects, a ratio of digestive enzyme to substrate is selected to ensure limited digestion. In some exemplary embodiments, a ratio of digestive enzyme to substrate is less than about 1:100, less than about 1:200, less than about 1:300, less than about 1:400, less than about 1:500, less than about 1:600, less than about 1:700, less than about 1:800, less than about 1:900, less than about 1:1000, less than about 1:2000, less than about 1:3000, less than about 1:4000, less than about 1:5000, less than about 1:6000, less than about 1:7000, less than about 1:8000, less than about 1:9000, less than about 1:10000, about 1:400, about 1:1000, about 1:2500, or about 1:10000.

As used herein, the term “protein reducing agent” or “reduction agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof. A reducing step may be performed in sequence with or concurrent with other sample preparation steps. For example, a reducing step and a denaturing step may be performed simultaneously (by adding a reducing agent while incubating a sample at high temperatures) so that cysteines exposed to the solvent by denaturing can be accessed by the reducing agent.

As used herein, the term “liquid chromatography” refers to a process in which a biological and/or chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, the sample or eluate can be subjected to any one of the aforementioned chromatographic methods or a combination thereof.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

The mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

SRM/MRM/Selected-ion monitoring (SIM) is a method used in tandem mass spectrometry in which an ion of a particular mass is selected in the first stage of a tandem mass spectrometer and an ion product of a fragmentation reaction of the precursor ion is selected in the second mass spectrometer stage for detection. Examples of triple quadrupole mass spectrometers (TQMS) that can perform MRM/SRM/SIM include but are not limited to QTRAP® 6500 System (Sciex), QTRAP® 5500 System (Sciex), Triple QTriple Quad 6500 System (Sciex), Agilent 6400 Series Triple Quadrupole LC/MS systems, and Thermo Scientific™ TSQ™ Triple Quadrupole system.

In addition to MRM, the choice of peptides can also be quantified through Parallel-Reaction Monitoring (PRM). PRM is the application of SRM with parallel detection of all transitions in a single analysis using a high-resolution mass spectrometer. PRM provides high selectivity, high sensitivity and high-throughput to quantify selected peptides (Q1), and hence quantify proteins. Multiple peptides can be specifically selected for each protein. PRM methodology can use the quadrupole of a mass spectrometer to isolate a target precursor ion, fragment the targeted precursor ion in the collision cell, and then detect the resulting product ions in the Orbitrap mass analyzer. PRM can use a quadrupole time-of-flight (QTOF) or hybrid quadrupole-orbitrap (QOrbitrap) mass spectrometer to carry out the identification of peptides and/or proteins. Examples of QTOF include but are not limited to TripleTOF® 6600 System (Sciex), TripleTOF® 5600 System (Sciex), X500R QTOF System (Sciex), 6500 Series Accurate-Mass Quadrupole Time-of-Flight (Q-TOF) (Agilent) and Xevo G2-XS QT of Quadrupole Time-of-Flight Mass Spectrometry (Waters). Examples of QObitrap include but are not limited to Q Exactive™ Hybrid Quadrupole-Orbitrap Mass Spectrometer (Thermo Scientific) and Orbitrap Fusion™ Tribrid™ (Thermo Scientific).

Non-limiting advantages of PRM include: elimination of most interferences; providing more accuracy and attomole-level limits of detection and quantification; enabling the confident confirmation of the peptide identity with spectral library matching; reducing assay development time since no target transitions need to be preselected; and ensuring UHPLC-compatible data acquisition speeds with spectrum multiplexing and advanced signal processing.

The mass spectrometer in the methods or systems of the present application can be, for example, an electrospray ionization mass spectrometer, nano-electrospray ionization mass spectrometer, or a triple quadrupole mass spectrometer, wherein the mass spectrometer can be coupled to a liquid chromatography system, wherein the mass spectrometer is capable of performing LC-MS (liquid chromatography-mass spectrometry) or LC-PRM-MS (liquid chromatography-parallel reaction monitoring-mass spectrometry) analyses. In some exemplary embodiments, the identification of peptides is performed using PRM-MS.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS2, can be performed by first selecting and isolating a precursor ion (MS1), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited to, identifying the protein, sequencing amino acids of the protein fragments, determining protein sequencing, quantifying the protein, locating post-translational modifications, identifying post translational modifications, or comparability analysis, or combinations thereof.

In some exemplary aspects, the mass spectrometer can use nanoelectrospray or nanospray ionization. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (matrixscience.com), Spectrum Mill (chem.agilent.com), PLGS (waters.com), PEAKS (bioinformaticssolutions.com), Proteinpilot (download.appliedbiosystems.com/proteinpilot), Phenyx (phenyx-ms.com), Sorcerer (sagenresearch.com), OMSSA (pubchem.ncbi.nlm.nih.gov/omssa/), X!Tandem (thegpm.org/TANDEM/), Protein Prospector (prospector.ucsf.edu/prospector/mshome.htm), Byonic (proteinmetrics.com/products/byonic) or Sequest (fields.scripps.edu/sequest).

It is understood that the present invention is not limited to any of the aforesaid protein(s), therapeutic protein(s), antibody(s), recombinant protein(s), host-cell protein(s), protein pharmaceutical product(s), sample(s), AAV(s), virus(es), serotype(s), vector(s), protein alkylating agent(s), protein denaturing agent(s), protein reducing agent(s), digestive enzyme(s), chromatographic method(s), mass spectrometer(s), database(s), bioinformatics tool(s), pH range(s) or value(s), temperature(s), or concentration(s), and any protein(s), therapeutic protein(s), antibody(s), recombinant protein(s), host-cell protein(s), protein pharmaceutical product(s), sample(s), AAV(s), virus(es), serotype(s), vector(s), protein alkylating agent(s), protein denaturing agent(s), protein reducing agent(s), digestive enzyme(s), chromatographic method(s), mass spectrometer(s), database(s), bioinformatics tool(s), pH, temperature(s), or concentration(s) can be selected by any suitable means.

The present invention will be more fully understood by reference to the following examples. They should not, however, be construed as limiting the scope of the invention.

### EXAMPLES

Materials. Adeno-associated viral samples were purchased from Charles River. Dithiothreitol (DTT), iodoacetamide (IAM/IAA) and Tris(2-carboxyethyl)phosphine hydrochloride (TCEP) were purchased from Thermo Scientific (Rockford, IL). Sequencing grade modified trypsin was purchased from Promega (Madison, WI), Cat #: V511A. Tris-HCl buffer (pH 7.5) was purchased from Invitrogen (Carlsbad, CA). Urea and acetic acid were purchased from Sigma-Aldrich (St. Louis, MO). LC-MS grade solvents were purchased from Honeywell (Charlotte, NC).

100 mM Tris HCl (pH 7.5) was prepared by diluting 10 mL of IM Tris-HCl (pH 7.5) into Milli-Q water and the total volume was brought to 100 mL. 50 mM TCEP was prepared by adding 90 μL of 0.1 M Tris HCl to 10 μL of 0.5 M TCEP to make 5 mM TCEP. To prepare IM DTT in 0.1 M Tris pH 7.5, 50 μL of 0.1 M Tris pH 7.5 was added to 7.7 mg/vial DTT, and was prepared fresh for each use. To prepare 100 mM DTT in 0.1 M Tris pH 7.5, 10 μL of IM DTT was mixed with 90 μL of 0.1 M Tris pH 7.5. To prepare 8 M urea denaturing solution (8M urea, 5 mM DTT, 0.1 M Tris-HCl), 0.48 g urea was dissolved in 630 μL of 0.1 M Tris-HCl, pH 7.5. 5 μL of IM DTT was added to make a final volume of 1 mL. The solution was vortexed until completely dissolved and made fresh for each use. To prepare 0.25 M IAM, 201.1 μL of 50 mM Tris-HCl (pH 7.5) was added to an IAM vial. To prepare 50 mM IAM, 10 μL of 0.25 M IAM was mixed with 40 μL of 50 Mm Tris-HCl (pH 7.5). To prepare 5% TFA solution, 1 mL of TFA was added into 19 mL of Milli-Q water in a 50 mL Pyrex glass bottle and mixed well. This solution was stored at room temperature for up to 3 months.

Mobile Phase A (0.1% FA in Milli-Q water) was prepared by adding 1 mL of FA to 1 L of Milli-Q water in a 2 L Pyrex glass bottle. The bottle was inverted 3-4 times to mix. The solution was stored at room temperature for up to 3 months. Mobile Phase B (0.1% FA in ACN) was prepared by adding 1 mL of FA to 1 L of Milli-Q water in a 2 L Pyrex glass bottle. The bottle was inverted 3-4 times to mix. The solution was stored at room temperature for up to 3 months. 5 mM acetic acid solution was prepared by adding 14.25 μL of glacial acetic acid to 50 mL of Milli-Q water.

Mild Denaturation/Differential Digestion. Approximately 2.5 μg of AAV sample was diluted to 100 μL with 100 mM Tris-HCl buffer at pH 7.5. Each sample was reduced with 5 mM TCEP or DTT at different denaturation temperatures for 30 minutes. 6 L of 50 mM IAM was added to the sample mixture and incubated in the dark for 30 minutes. 10 μL of trypsin was added at various concentrations and the samples were incubated at 37° C. for 2, 4, or 18 hours. After digestion, the samples were centrifuged at 12,000 rpm for 20 minutes. Around 110 μL of supernatant was transferred to an Eppendorf tube and acidified with 5 L of glacial acetic acid. The remaining solution (pellet) was digested per the direct digestion protocol described below to analyze the encapsulated or difficult to digest HCPs.

Direct Digestion. Approximately 2.5 μg of AAV samples were denatured and reduced with 8 M urea in 0.1 M Tris/HCl, pH 7.5, 5 mM DTT at 80° C. while shaking at 650 rpm for 30 minutes. The denatured and reduced samples were cooled down to room temperature and alkylated with 6 μL of 50 mM IAM in the dark for 30 minutes. Lyophilized sequence grade trypsin was reconstituted in 50 mM Tris/HCl and 10 μL of 0.0125 μg/μL solution was mixed with the reduced and alkylated sample at 37° C. in the dark with shaking at 650 rpm in a thermomixer for 4 hours. After digestion, 2 μL of glacial acetic acid was added to stop the reaction, and the samples were vortexed for 3-5 seconds. The total volume was 20 μL. 5 μL of sample (1 μg) was injected on LC-MS.

LC-MS/MS analysis. The digested samples were analyzed using an UltiMate 3000 RSLCnano system (Thermo Scientific) coupled to an Exploris-480 mass spectrometer (Thermo Scientific). The RSLCnano system was equipped with an Acclaim PepMap 100 75 μm×2 cm trap column and an Acclaim PepMap 75 μm×25 cm C18 analytical column. The mobile phase A was 0.1% formic acid in water and mobile phase B was 0.1% formic acid in 80% acetonitrile. The flow rate was 300 nL/min and linear LC gradient was set as follows: 5% B at 0-6 min, 32% B at 85 min, 20% B at 20 min, 36% B at 125 min, 95% B at 133-141 min, 5% B at 143-150 min. The Mass Spectra data acquisition was operated using top 10 DDA method. MS1 scan range was 380-1600 m/z at 60 k resolution (m/z 400). The MS/MS isolation window was set to 1.4 m/z and normal collision energy (NCE) set to 28. Minimum automatic gain control (AGC) was set to 1e4 with maximum IT of 20 ms.

Data analysis. The raw files were searched against the Uniprot Homo sapiens and virus proteome database using SEQUEST in Proteome Discoverer 2.4 (Thermo Scientific). Precursor mass tolerance was set to 10 ppm and fragment ion mass tolerance was set to 0.02 Da. Trypsin was set as the digestion enzyme. Methionine oxidation was set as dynamic modification and cysteine alkylation was set as static modification. Proteins with a minimum of two unique peptides detected and peptide length >6 amino acids with high-quality MS/MS spectra were filtered as true positive. Target FDR was set to 1%. At least two unique peptides were required for true identification. Normalization mode and scaling mode was set to none. Protein abundance calculation was based on summed abundances of identified peptides.

### Example 1. Comparison of Digestion Methods

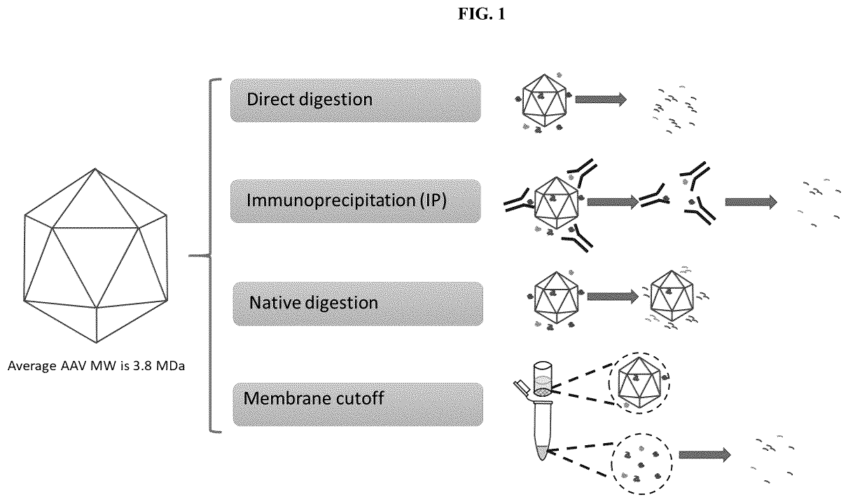

Current methods for HCP identification include direct digestion, immunoprecipitation, native digestion, and the use of molecular weight cutoff filters, as shown in FIG. 1. However, there are several challenges and shortcomings with the current methods for HCP identification, especially while profiling AAVs for HCP impurities. Some of the challenges and shortcomings include, for example, sample limitation and batch-to-batch variation during production.

The direct digestion method involves treating a sample with a denaturing reagent, heating to an elevated temperature, and then treating a sample with a digestive enzyme. While HCP impurities have been identified with this process, this process also denatures the AAV into its respective capsid proteins as well, leading to a digested sample with a high ratio of peptides from viral proteins compared to HCPs, which makes sensitive detection of HCPs difficult.

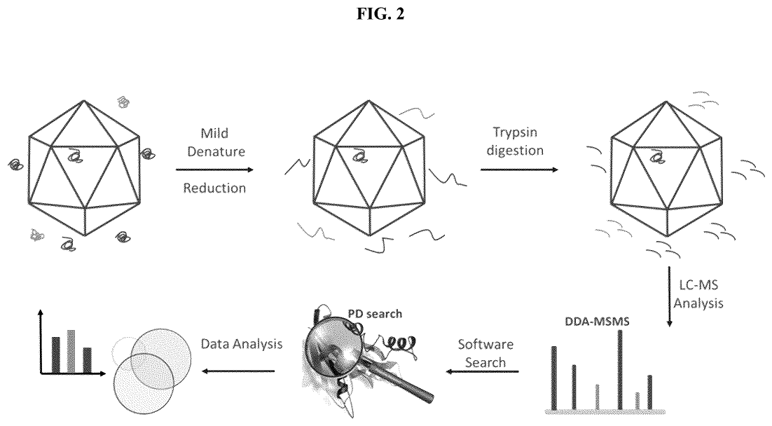

An alternative approach was developed, taking advantage of the fact that AAV particles have higher thermal stability than contaminating HCPs. The novel method, called “limited digestion” or “differential digestion,” includes subjecting a sample to mild denaturing conditions that preferentially denature HCPs but leave AAV vectors intact, thus increasing the sensitivity of HCP detection. AAV is more resistant to trypsin digestion than HCPs, and because AAV does not contain any disulfide bonds, reduction reagents would only break the disulfide bonds of HCPs while leaving AAV unaffected. By leveraging these unique characteristics, the differential digestion method involving mild denaturation and reduction before enzymatic digestion, followed by removal of the intact AAV through membrane filtration or centrifugation, and subsequent proteomic analysis of the supernatant ensures minimal digestion of the AAV while the residual HCPs are efficiently digested. Therefore, the sensitivity of HCP detection is increased by the methods of the present invention. Furthermore, the differential digestion conditions can be easily applied to various AAV serotypes. A subsequent digestion step preferentially produces peptides from the denatured HCPs and not the folded AAV capsid proteins, leading to an effective enrichment of HCPs in a sample, as illustrated in FIG. 2.

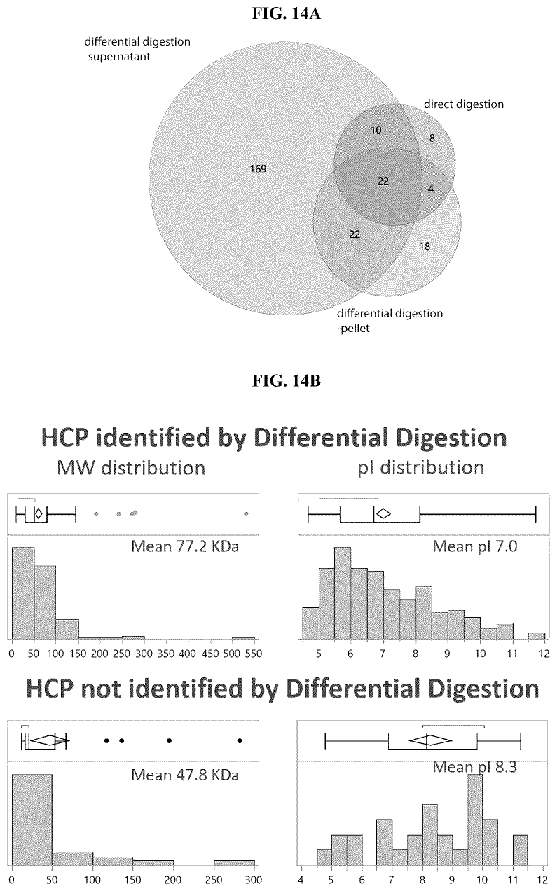

The differential digestion method was compared to the direct digestion method using a commercially available AAV1 sample. The total AAV peptide intensity under differential digestion conditions was 1.50c9, a value markedly lower than the 2.85c¹⁰ observed under the direct digestion conditions, thereby considerably decreasing the dynamic range. The intensity of two individual AAV peptides was also compared (FIG. 15) to confirm the limited digestion of AAV under differential digestion conditions. In a direct digested AAV1 sample, a peptide peak with an m/z of 810.91 showed an intensity of 1.18e⁸. The intensity of the same peptide decreased to 4.90e⁶ under differential digestion, representing a decrease by a factor of 25 (FIG. 15). Another AAV peptide with a sequence “LINNNWGFRPK” showed an intensity of 2.12e⁷ after direct digestion and an intensity of 3.12e⁶ after differential digestion, thus indicating a decrease by a factor of 6 (FIG. 15).

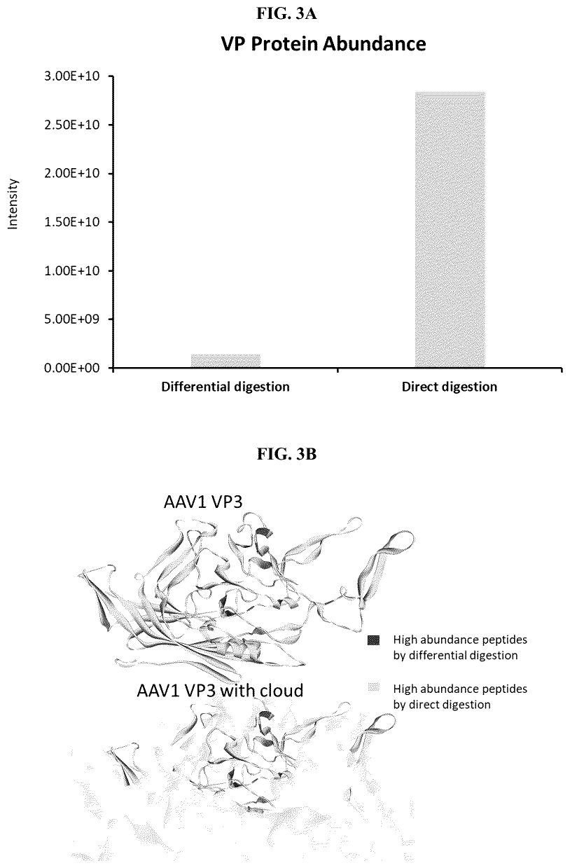

Analysis using direct digestion yielded a measured abundance of viral proteins in the sample that was several fold higher than the abundance measured when using differential digestion, as shown in FIG. 3A.

To further correlate the AAV digestion efficiency of individual peptide to high order structure, the accessibility of viral capsid protein to trypsin is mapped onto the AAV1 capsid cryoEM structure (PDB 8FQ4) (FIG. 3B (top panel) and FIG. 3B (bottom panel)). Trypsin proteolytic sites with identified and quantified peptides are highlighted in either red (for higher abundance peptides by differential digestion) or yellow (for higher abundance peptides by direct digestion). FIG. 3B (top panel) shows the AAV1 VP3 structure of a single subunit in cartoon representation. Moreover, FIG. 3B (bottom panel) shows the same subunit in cartoon representation, and also displays all other surrounding subunits in an intact capsid in cloud representation, to visualize regions of the viral protein buried in the inter-subunit interface. The results demonstrated that the peptides with higher abundance after differential digestion were mostly solvent accessible, whereas the higher abundance peptides detected after direct digestion were mostly shielded by the cloud and buried in the interface. The two peptides, as shown in FIG. 15, were confirmed to be buried in the interface of this cryoEM structure. This pattern was consistent with the fact that differential digestion is a mild condition and the inability of low abundance trypsin to access buried regions. More importantly, under the initial differential digestion conditions, more HCPs are identified after differential digestion than direct digestion. This result confirmed that the mild denaturation conditions used for differential digestion substantially preserved the folded state of AAV proteins, leading to a depletion of AAV proteins from the sample.

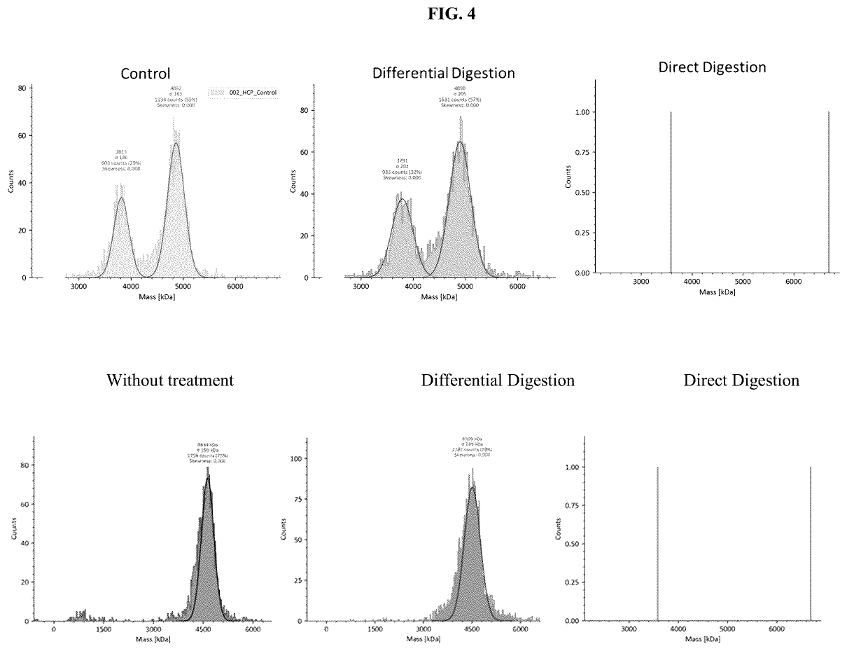

Furthermore, mass photometry was used to show that the method of differential digestion did not fully degrade AAV particles. FIG. 4 (top panel) shows the relative abundance of AAVs with full or empty capsids following differential digestion, direct digestion, or control treatment, using mass photometry. The profiles of intact AAV capsid before treatment (FIG. 4, bottom panel) and after trypsin treatment under differential digestion conditions (FIG. 4 bottom panel) were found to be similar, and most of the AAV capsid remained intact after digestion. In contrast, under direct digestion conditions with a denaturing reagent and heating at 80° C. (FIG. 4, bottom panel), the AAV peak at 4.5 MDa disappeared, thus indicating that the AAV capsid was disrupted under the direct digestion conditions. AAVs subjected to differential digestion showed a similar signal compared to the control group, while AAVs subjected to direct digestion showed a single sharp peak, suggesting complete degradation of the AAV.

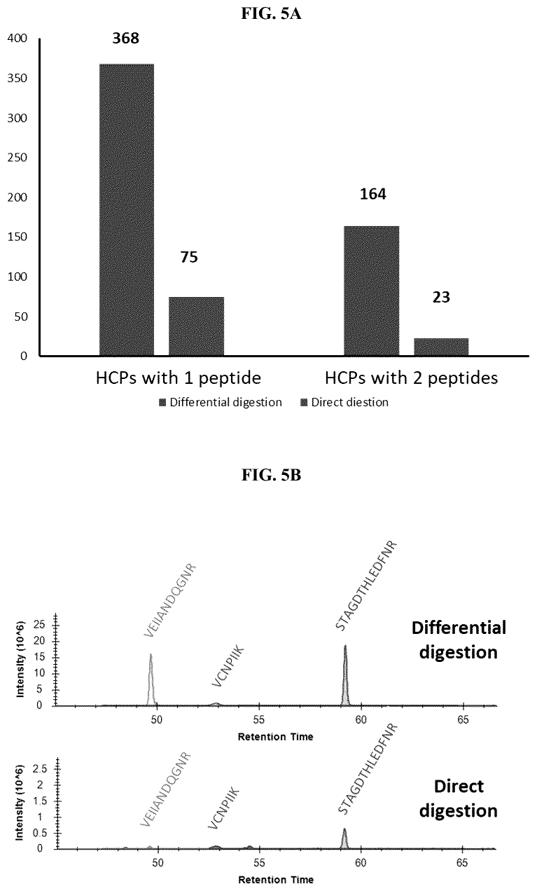

FIG. 5A shows a comparison of the number of HCPs identified with 1 peptide or 2 peptides after differential or direct digestion. The differential digestion method showed a greatly improved ability to identify HCPs in both categories. The sensitivity of the differential digestion as compared to the direct digestion is further demonstrated in FIG. 5B. Over a 10-fold increase in peptide intensity was observed for the same protein for the differential digestion method compared to the direct digestion method. To maximize HCP detection, additional experiments were conducted to optimize the conditions for differential digestion.

### Example 2. Parameter Optimization

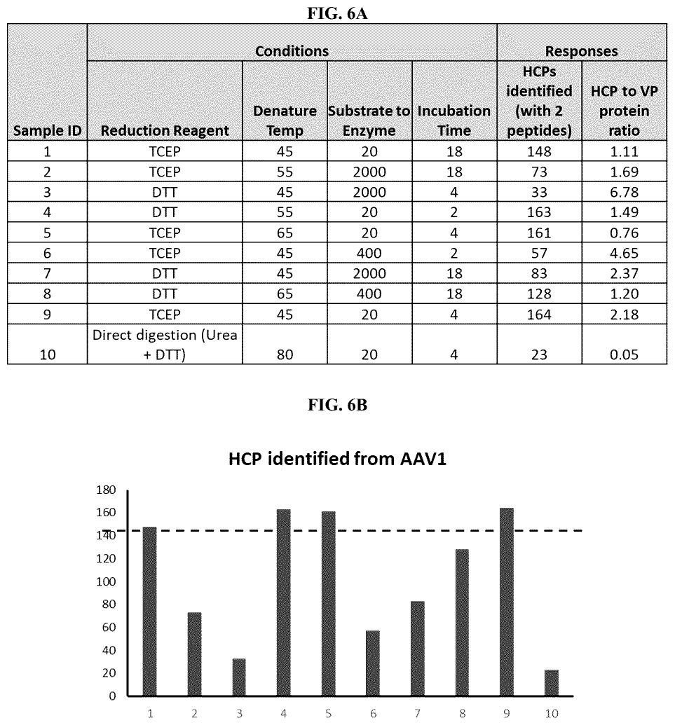

Based on the effectiveness of the differential digestion method for HCP identification as shown in Example 1, this method was further optimized. A Design of Experiments approach was used to elucidate the relationship between method parameters and outcomes in order to determine critical factors. The method was conducted with varying denaturation temperatures, varying ratios of trypsin enzyme:substrate for digestion, varying incubation times for digestion, and various reducing agents. The combinations of parameters tested are shown in FIG. 6A. A denaturation temperature of 45° C., a ratio of 1:20 of enzyme to substrate and a four-hour incubation was found to be most effective in terms of the number of HCPs identified and a low HCP to viral capsid protein (VP) ratio, as shown in FIG. 6A, sample 9. In FIGS. 6B and 6C, the number of HCPs identified and the ratio of HCP to VPs are shown for each of the 10 samples tested in FIG. 6A, respectively.

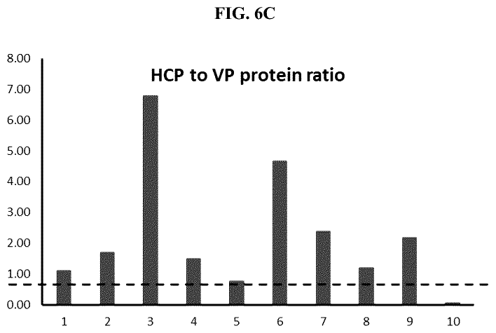

The relationships between the method parameters (reducing reagent, denaturation temperature, enzyme:substrate ratio and incubation time) and the method outcomes (number of HCPs identified and HCP to VP protein ratio) were modeled using a prediction profiler to determine the optimal conditions for the differential digestion based on specific desirability entered, as shown in FIG. 7. Based on the variables tested, the final parameters selected were TCEP as a reducing agent, 45° C. as a denaturation temperature, 1:20 as an enzyme to substrate ratio, and a 4-hour incubation time. These optimized conditions were used for further experiments.

### Optimization of Enzyme Concentration for Differential Digestion

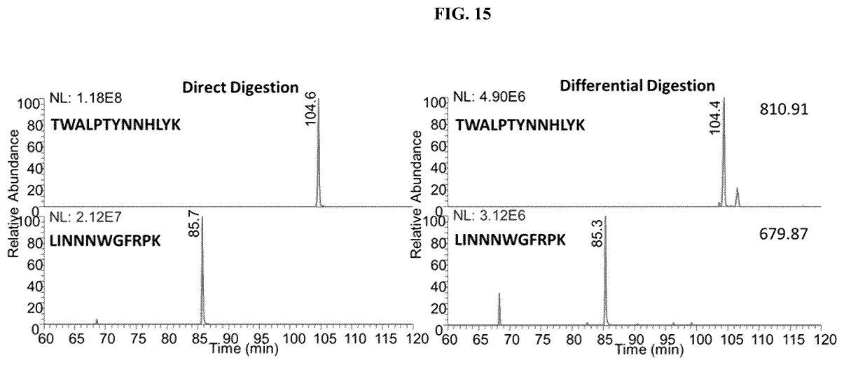

The enzyme concentration of differential digestion was essential to ensure method's sensitivity, given that this method was based on the hypothesis that AAV is less accessible than HCPs to trypsin under mild denaturation and reduction conditions. To determine the optimum digestion condition, AAV/trypsin ratios of 2000:1, 400:1 and 20:1 was evaluated after denaturation and reduction with 5 mM DTT at 45° C. As shown in FIG. 16, more HCPs were identified at an AAV:trypsin ratio of 20:1 than ratios of 400:1 or 2000:1. The original native digestion method for mAb HCP analysis used a 400:1 substrate to enzyme ratio. Subsequent studies have further optimized the native digestion condition and used an ultra-low 10000:1 substrate to enzyme ratio to achieve optimal HCP identification. When the native digestion condition, which used a 2000:1 substrate to enzyme ratio, was initially applied for AAV HCP profiling, fewer HCPs were identified than those observed with direct digestion, indicating a higher enzyme concentration is preferred for AAV HCP analysis.

A significantly greater number of HCPs were identified when greater amounts trypsin was used (97 HCPs identified at high trypsin concentration versus 6 HCPs identified at low trypsin concentration). The difference in the preference for trypsin concentration between AAV and mAb might have been due to their distinct physical properties. AAV is more resistant to trypsin digestion than mAb because of AAV's high order structure and high molecular weight. Additionally, because of limited AAV quantity, only several micrograms of AAV sample was subjected to HCP analysis, as compared with several milligrams of mAb sample. When limited digestion was conducted, both drug substances and HCPs underwent partial digestion. mAb HCP analysis by native digestion, compared with direct digestion, allowed for the injection of approximately 40 times more samples. Therefore, limited digestion of HCPs under native conditions may not significantly impact the number of HCPs detected. In contrast, AAV HCP analysis is limited by the sample amount, thus necessitating a higher level of trypsin to ensure sufficient digestion efficiency of HCP. The optimal enzyme to AAV ratio of 1:20 was applied in subsequent optimization.

### Optimization of Denaturation and Reduction Conditions for Differential Digestion

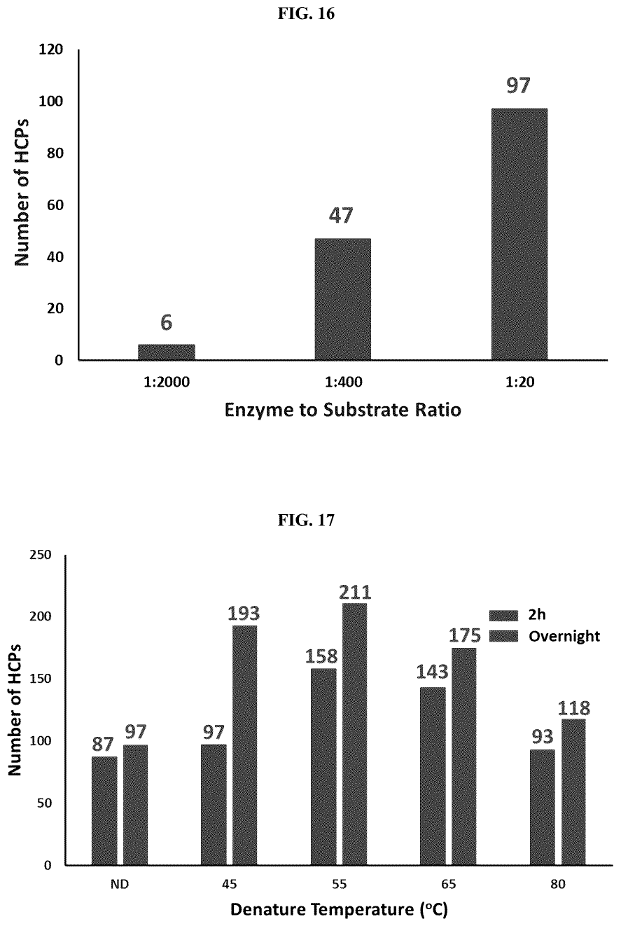

Another crucial parameter in the differential digestion workflow is the denaturing temperature. Ideally, this temperature should effectively denature the HCPs while maintaining the integrity of the AAV capsid. The literature has shown that the capsid melting temperature for all AAV serotypes is above 65° C. Therefore, three denaturing temperatures, 45° C., 55° C., and 65° C., in addition to no heating and heating at 80° C. were evaluated. As depicted in FIG. 17, the differential digestion methods conducted at varying denaturing temperatures led to identification of more HCPs than the native digestion method. Denaturing the samples at 55° C. was more effective for HCP identification, resulting in the detection of 158 HCPs with more than two unique peptides after 2 hours of digestion. A temperature of 45° C. was not adequate to denature the HCPs and ensure efficient enzymatic digestion, because only 97 HCPs were identified. Similarly, 93 HCPs were identified under native digestion conditions with an optimal enzyme to substrate ratio of 1:20. In contrast, the AAV capsid was disrupted and further digested when the denaturation temperature reached 65° C. or above, resulting in identification of fewer HCPs.

Because of the absence of disulfide bonds in AAV, the presence of reducing agents did not affect the integrity of AAV. The effects of two reducing agents, DTT and TCEP, on HCP identification were also assessed. DTT performed better than TCEP, although the difference was not significant. Consequently, DTT was chosen as the reducing agent for subsequent studies.

### Optimization of Incubation Time of Differential Digestion

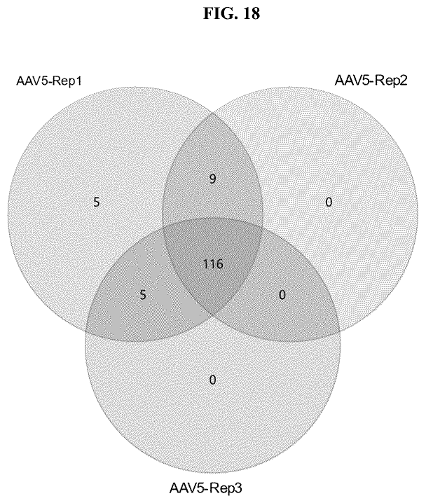

To further improve HCP identification, the incubation time was optimized by comparing 2-hour digestion and overnight digestion (FIG. 18). Although minimal improvement in HCP identification was achieved with an extended digestion time under native conditions (97 HCPs with overnight digestion vs 87 HCPs at 2 h digestion), overnight digestion led to a significantly higher number of HCPs identified under differential digestion conditions. Using a denaturation temperature of 55° C., 211 HCPs were identified after overnight incubation, as compared with 158 HCPs identified after differential digestion. This difference is attributable to the comparable digestion efficiency of HCPs and AAV under native conditions. However, differential digestion increased the digestion efficiency of HCPs over AAV, because HCPs were denatured and reduced. Longer digestion times therefore further improved HCP identification under differential digestion conditions. Consequently, overnight digestion was chosen for all subsequent experiments.

### Reproducibility of the Method

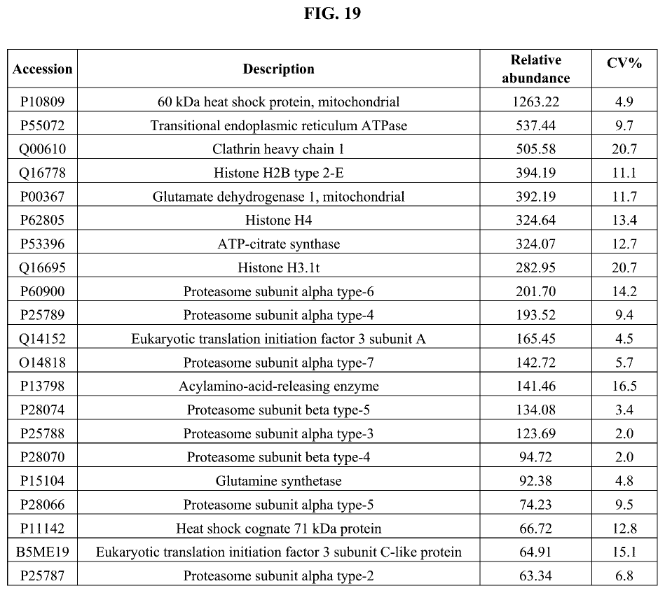

Method reproducibility and robustness are crucial for HCP analysis. The reproducibility of the workflow was evaluated by preparing three AAV5 samples under optimal conditions. A total of 116 HCPs, accounting for 86% of the total detected HCPs, were consistently detected in the three preparations, as shown in FIG. 18. The few proteins identified in only one or two of the three preparations were low abundance proteins, and these results were likely to be attributable to the technical variability of LC-MS. The relative abundance of the top 20 HCPs in AAV5 are summarized in FIG. 19. The triplicate sample preparation revealed minor variations in HCP quantitation (CV=2-20.7%), thus highlighting the good reproducibility of the methods.

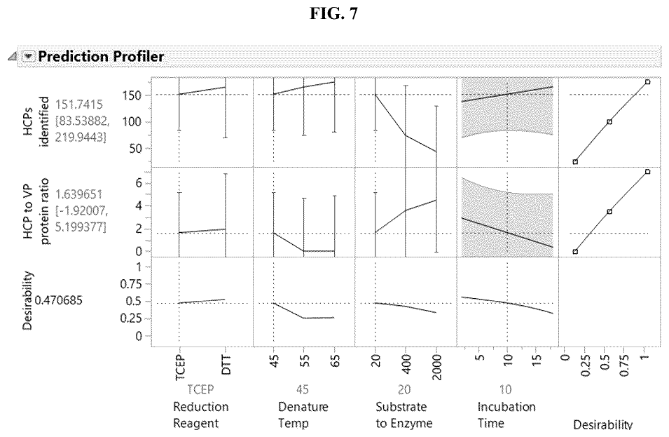

Over three preparations of AAV samples, 85% of HCPs were consistently identified, as depicted in FIG. 8A. Additionally, the measured abundance of each particular HCP was consistent across replications of the method, as shown in FIG. 8B.

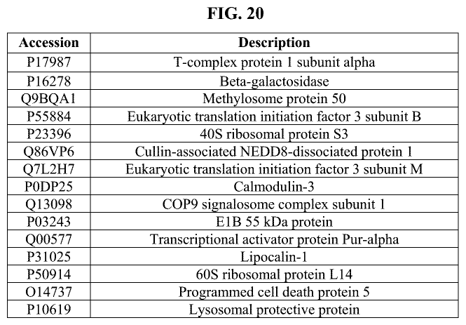

Because intact AAV was removed after digestion, HCPs that were difficult to digest or were encapsulated were removed in this step. To test whether encapsulated HCPs or undigested HCPs were present, the membrane filtered intact AAV5 were subjected to direct digestion. The identification of 15 additional HCPs (FIG. 20) in the remaining intact AAV5 suggested the potential presence of encapsulated HCPs.

### Example 3. Case Studies Across AAV Serotypes

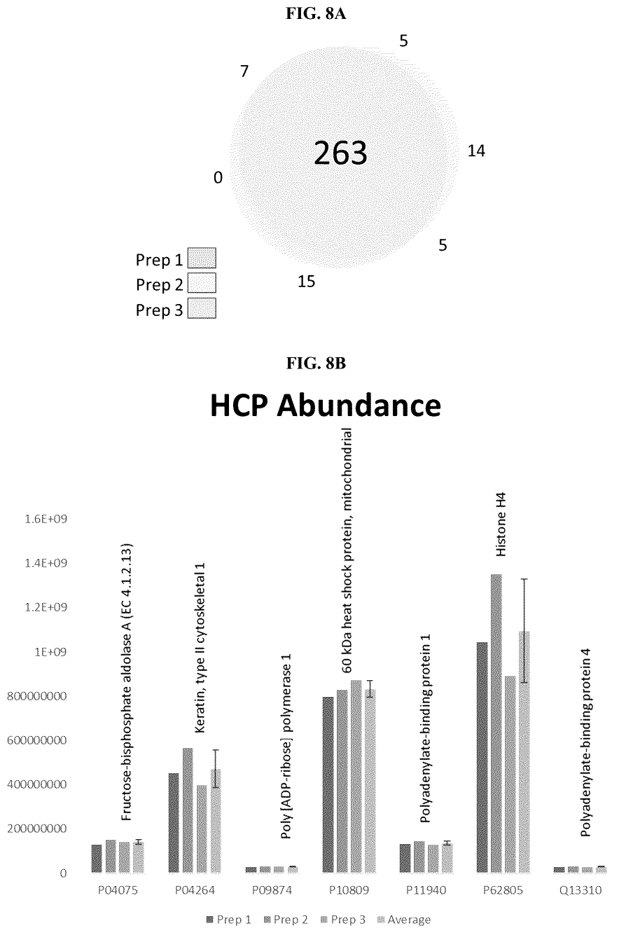

The differential digestion method of the present invention was used to analyze HCPs for three in-house produced AAV drug substance (DS) samples. The number of HCPs identified using differential digestion were compared to direct digestion analysis for AAV1, AAV8 and AAV9, as shown in FIG. 9A. The quantification includes only HCPs identified with more than 2 unique peptides. The results from differential digestion with two different injection amounts, 1 μg or 2 μg, were also compared. The differential digestion method of the present invention was capable of identifying a greater number of HCPs compared to conventional direct digestion. This is particularly evident for AAV9, as 5 times more HCPs were identified using differential digestion.

Unlike the direct digestion method, the reduced number of peptides produced using differential digestion meant that the LC-MS peaks of HCPs were not saturated when injecting 1 μg, and the injection amount could be increased to 2 μg. The number of HCPs identified in each of the samples was further increased when the injection amount was doubled, as shown in FIG. 9A. FIG. 9B shows extracted ion chromatograms (XIC) for variable injection amounts of differentially digested AAVs. With the increase in the amount of sample injected, an improvement in the extracted peak quality, increased peak intensity and identification confidence was observed.

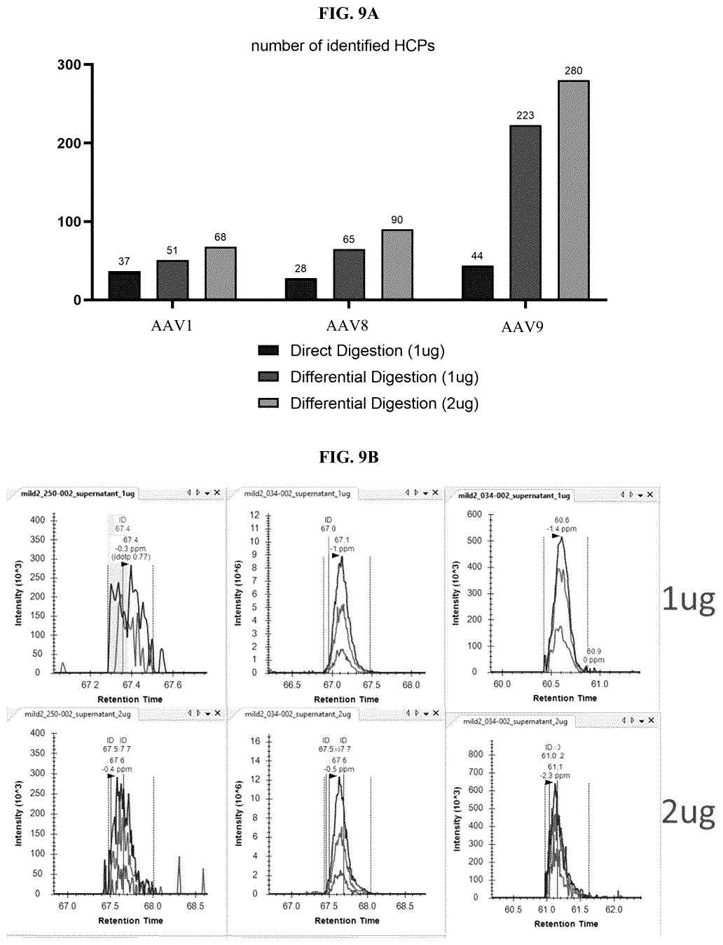

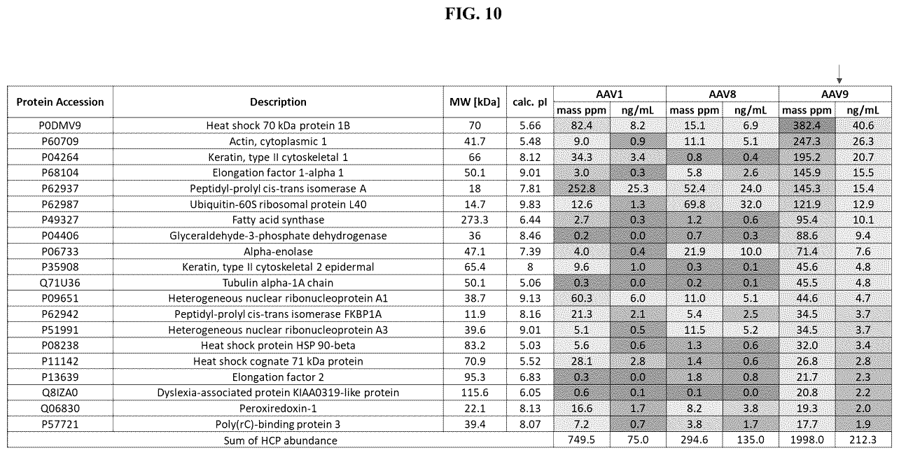

HCP profiling using direct digestion or differential digestion was carried out on each of the AAV1, AAV8, and AAV9 samples, as shown in FIG. 10 and FIG. 11 respectively. In total, there were 53 HCPs identified across the 3 samples. FIG. 10 shows the top 20 abundant HCPs, sorted by the HCP abundance in AAV9 after direct digestion. FIG. 11 shows the top 20 abundant HCPs, sorted by the HCP abundance in AAV9 after differential digestion. The quantitation is based on averaging the XIC peak area of the top 3 peptides to the average abundance of the top 3 peptides from viral proteins. The most abundant proteins identified were ribonucleoproteins, and their abundance contributes to half of the total HCP abundance. Identified HCPs and their abundances vary based on AAV serotype and the digestion carried out.

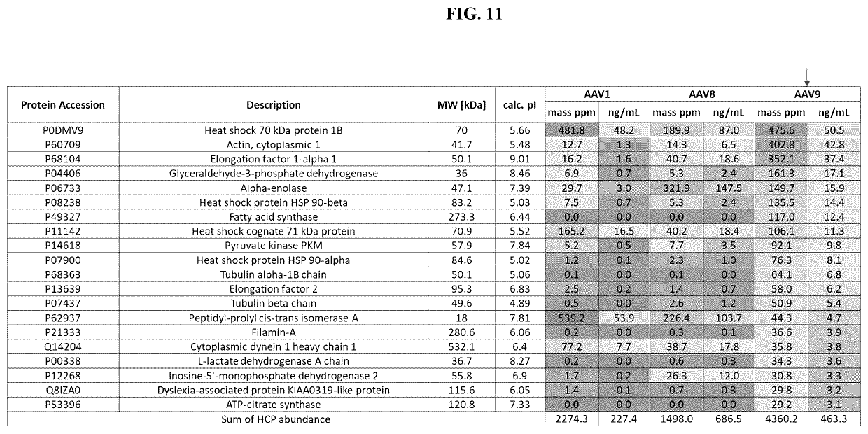

Some particular proteins of interest that were identified using direct digestion or differential digestion are shown in FIG. 12. The first two proteins, rep52 and rep78, are associated with the helper virus in the production of the viral vector. Rep52 was not identified by direct digestion and rep78 was identified with low abundance in the direct digestion method, but both proteins were enriched and identified by the differential digestion method of the present invention. Dyslexia-associated protein KIAA0319-like protein is a receptor for adeno-associated virus and is involved in adeno-associated virus infection through the endocytosis system. Only 1 unique peptide was identified by direct digestion, while 10 unique peptides were identified by the differential digestion method of the present invention. Glyceraldehyde-3-phosphate, Alpha-enolase and Heat shock 70 kDa protein 1B are commonly identified as top abundant HCPs in AAV samples, and they are all cytoplasm component related proteins. Due to their high similarity with sheep protein, an ELISA assay may be inefficient at detecting them, while they are identifiable using MS. Using the differential digestion method of the present invention, they were identified in greater quantity compared to the direct digestion method. In general, the method of the present invention improved identification of non-viral protein impurities present in an AAV sample.

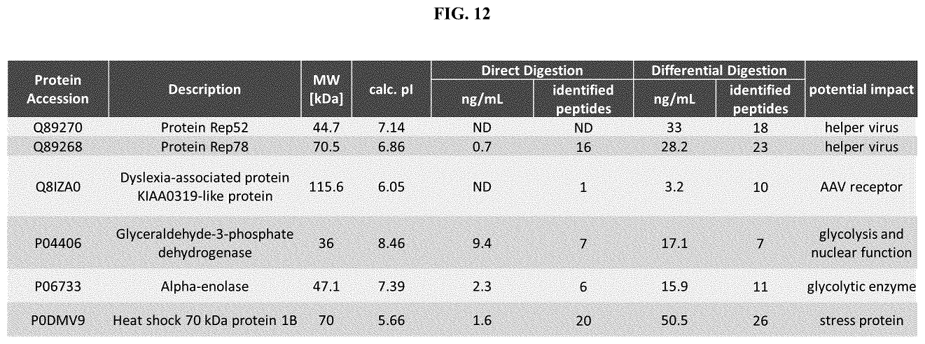

During AAV production, some HCPs may be encapsulated in the capsid. Using the differential digestion method described above, the capsid was kept intact and encapsulated HCPs remained enclosed and protected from reduction, denaturation and digestion. In order to analyze the HCPs encapsulated in the capsid of AAVs, an additional method was developed for analyzing encapsulated non-viral proteins, as shown in FIG. 13. Upon completion of the differential digestion, the sample was centrifuged to separate AAVs (and any encapsulated HCPs) from other components in the sample, for example free non-viral proteins, to form an enriched AAV sample. This separation step of the present invention allows for the centrifugal force to separate particles based on their size, shape, and density. This forces the heavier capsids into a pellet at the bottom of the tube, and the lighter material, such as unbound “free” proteins and impurities to remain in solution. A denaturation step at high heat was performed to denature the capsid as well as any encapsulated HCPs. Trypsin digestion was then used to produce a peptide digest of the capsid proteins and formerly encapsulated HCPs, and the peptide digest was subjected to LC-MS analysis. This allowed for the differentiation of HCPs encapsulated in the capsid versus those HCPs which are free in the supernatant.

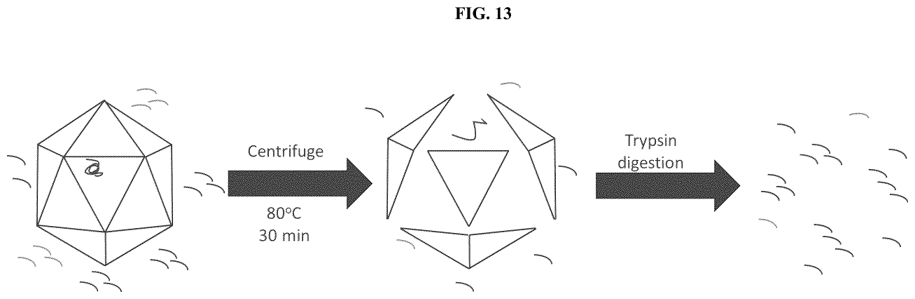

A comparison of the number of HCPs identified using the earlier-described differential digestion method (labeled here as differential digestion supernatant), identified using digestion of the AAV pellet following differential digestion (labeled here as differential digestion pellet), and identified using direct digestion is depicted in FIG. 14A. The highest number of HCPs were identified in the supernatant following differential digestion, which covered the majority of all HCPs identified by each of the methods. The majority of the proteins identified by direct digestion were also identified by differential digestion. While the majority of the proteins identified in the digestion of the AAV pellet following differential digestion were previously identified in the supernatant following differential digestion, there were 18 unique HCPs identified in the pellet, demonstrating that the present method for further analysis of encapsulated HCPs may be useful for a complete analysis of both free and encapsulated HCPs, or a comparison between the two classes of HCPs. FIG. 14B shows a comparison of the molecular weight and pI of HCPs identified or not identified by differential digestion. A significant difference in the molecular weight was observed for HCPs identified by differential digestion.

### HCP Analysis of Different AAV Serotypes

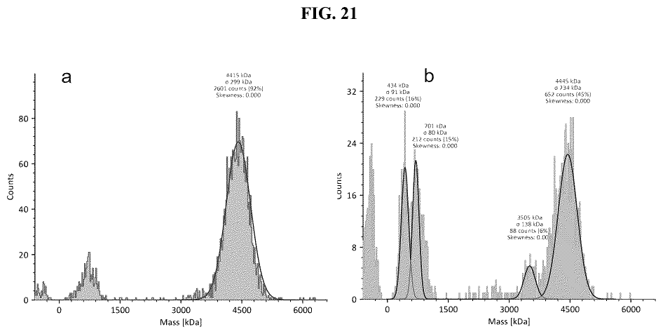

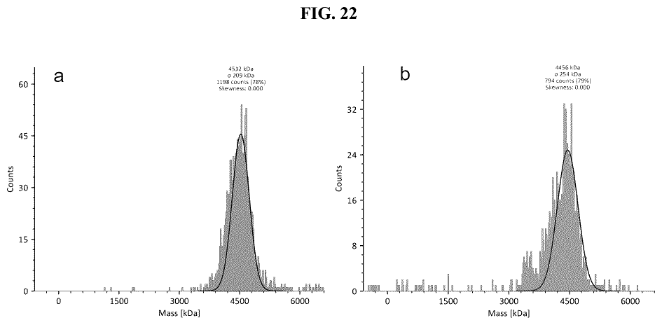

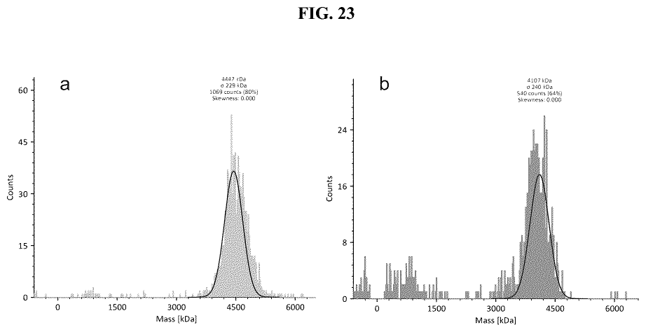

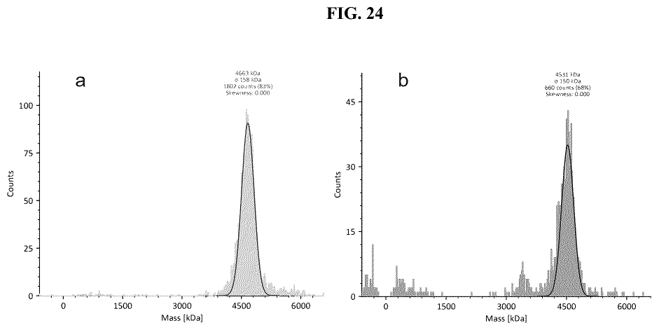

Different AAV serotypes yield different melting temperatures, which could potentially influence the effectiveness of the differential digestion workflow. To confirm that the differential digestion condition was applicable to different serotypes, AAV1, AAV2, AAV5, AAV6 and AAV9 were analyzed under this condition. As demonstrated in FIG. 21, FIG. 22, FIG. 23 and FIG. 24, the integrity of all AAV serotypes was preserved after differential digestion. This shows the AAV differential digestion method is a universal approach suitable for various AAV serotypes.

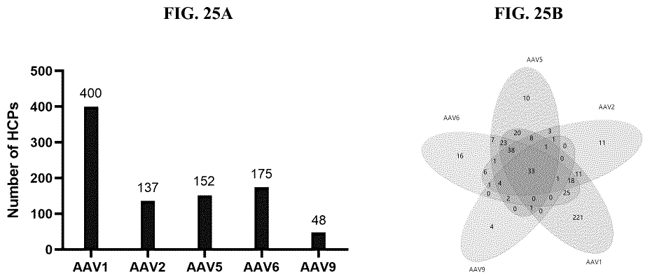

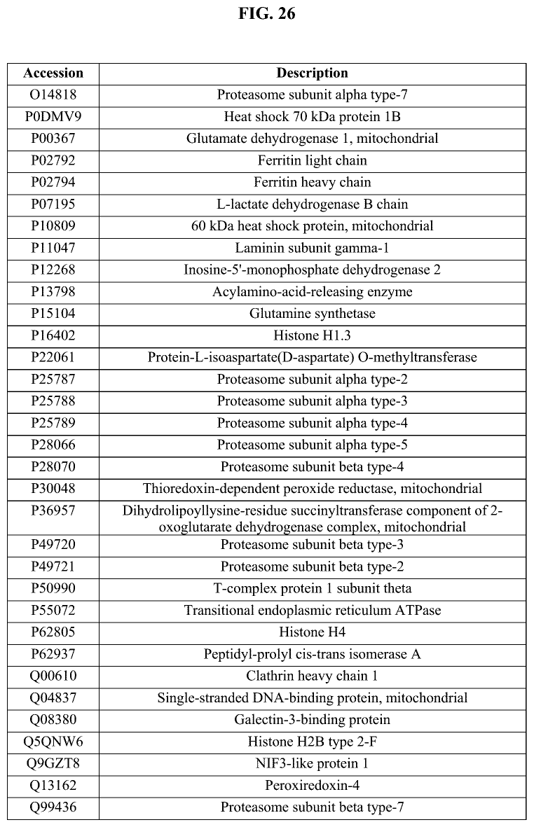

While there is extensive knowledge about HCP impurities in monoclonal antibodies (mAb), including the effects of problematic HCPs, information about HCPs in gene therapy products remains limited. In this study, comprehensive HCP profiling of five different serotypes were conducted. As shown in FIG. 25A, the number of identified HCPs from AAV1, AAV2, AAV5, AAV6 and AAV9 are 400, 137, 152, 175 and 48, respectively. The Venn diagram of AAV serotypes is shown in FIG. 25B. Among all of the HCPs identified, 33 of them (FIG. 26) are commonly identified in all serotypes. While 221 HCPs were uniquely identified in AAV1, fewer than 20 HCPs are uniquely identified in other AAV serotypes. Although this set of data shows higher number of HCPs identified in AAV1, it may not necessarily be a universal phenomenon as purification process was not considered in this study.

Several heat shock proteins were detected in all serotypes, and HSP60A is one of the most abundant HCPs detected. HSP90A protein, previously reported in HEK293-derived AAV2 and among the most abundant HCPs detected in HEK293-derived adenovirus vaccine, poses potential immunogenicity at high concentration. Multiple histone proteins, which were previously reported may pose immunogenicity risk, were present in all AAV serotypes. High abundance of proteasome was also identified in AAV, which plays a role in host-virus interaction. Y-box binding protein (YB1), which was previously identified in AAV and proved to have negative impact on AAV2 and AAV8 physical vector genome titers was identified in four different serotypes in this study.

In-depth HCP characterization of the AAV serotypes provides knowledge of HCP types and potential risk. Moreover, the information gained in this study may facilitate downstream purification process development to remove potential problematic HCPs. From a regulatory perspective, comprehensive study of HCPs may help to set appropriate thresholds of gene therapy products.

What is claimed is:

1. A method of identifying, quantifying, and/or characterizing at least one host cell protein (HCP) impurity in a sample containing AAV vectors, comprising:
(a) treating a sample containing at least one HCP impurity and at least one AAV vector to a reducing agent to produce a reduced sample;
(b) subjecting said reduced sample to mild denaturation conditions to produce a partially denatured sample;
(c) subjecting said partially denatured sample to enzymatic digestion conditions to produce a peptide digest; and
(d) subjecting said peptide digest to liquid chromatography-mass spectrometry (LC-MS) analysis to identify, quantify, and/or characterize said at least one HCP impurity.

2. The method of claim 1, wherein said reducing agent is selected from TCEP or DTT.

3. The method of claim 2, wherein said reducing agent is TCEP.

4. The method of claim 1, wherein said mild denaturation conditions comprise a temperature from about 35° C. to about 65° C.

5. The method of claim 4, wherein said temperature is about 35° C., about 40° C., about 45° C., about 50° C., about 55° C., about 65° C., about 70° C. or about 75° C.

6. The method of claim 1, wherein said enzymatic digestion conditions comprise contacting said partially denatured sample to at least one digestive enzyme, optionally wherein said at least one digestive enzyme comprises trypsin.

7. The method of claim 6, wherein said trypsin is present at an enzyme to substrate ratio of from 1:10 to 1:4000, about 1:20, about 1:400, or about 1:2000.

8. The method of claim 7, wherein in said enzyme to substrate ratio is about 1:20.

9. The method of claim 1, wherein a duration of said enzymatic digestion conditions is from 1 to 20 hours, about 2 hours, about 4 hours, or about 18 hours.

10. The method of claim 1, wherein an amount of said peptide digest injected on LC-MS is from about 1 μg to about 4 μg, about 1 μg, about 1.5 μg, about 2 μg, about 3 μg, or about 4 μg.

11. A method of identifying, quantifying, and/or characterizing at least one host cell protein (HCP) impurity in the capsid of an AAV vector, comprising:
(a) separating a sample including at least one HCP impurity in the capsid of an AAV vector from free HCPs to produce an enriched AAV vector sample;
(b) treating said enriched AAV vector sample to a reducing agent to produce a reduced sample;
(c) subjecting said reduced sample to denaturation conditions to produce a denatured sample;
(d) treating said denatured sample to enzymatic digestion conditions to produce a peptide digest; and
(e) subjecting said peptide digest to LC-MS analysis to identify, quantify, and/or characterize said at least one HCP impurity.

12. The method of claim 11, wherein said reducing agent is selected from TCEP or DTT.

13. The method of claim 12, wherein said reducing agent is DTT.

14. The method of claim 11, wherein said enzymatic digestion conditions comprise contacting said denatured sample to at least one digestive enzyme, optionally wherein said at least one digestive enzyme comprises trypsin.

15. The method of claim 11, wherein a duration of said enzymatic digestion conditions is from 1 to 20 hours, about 2 hours, about 4 hours, or about 18 hours.

16. The method of claim 11, wherein an amount of said peptide digest injected on LC-MS is from about 1 μg to about 4 μg, about 1 μg, about 1.5 μg, about 2 μg, about 3 μg, or about 4 μg.

17. A method of enriching and identifying HCP impurities in a sample comprising treating the sample with a reducing agent, followed by a denaturation step, followed by enzymatic digestion, and identification of said at least one enriched HCP using LC-MS.

18. The method of claim 17, wherein the sample includes a supernatant and a pellet.

19. The method of claim 17, wherein said reducing agent is selected from TCEP or DTT.

20. The method of claim 17, wherein said mild denaturation conditions comprise a temperature from about 35° C. to about 80° C.

21. The method of claim 20, wherein said temperature is about 35° C., about 40° C., about 45° C., about 50° C., about 55° C., about 65° C., about 70° C., about 75° C., about 80° C. or about 85° C.

22. The method of claim 17, wherein said enzymatic digestion conditions comprise contacting said denatured sample to at least one digestive enzyme, optionally wherein said at least one digestive enzyme comprises trypsin.

23. The method of claim 22, wherein said trypsin is present at an enzyme to substrate ratio of from 1:10 to 1:4000, about 1:20, about 1:400, or about 1:2000.

24. The method of claim 23, wherein said enzyme to substrate ratio is about 1:20.

25. The method of claim 17, wherein a duration of said enzymatic digestion conditions is from 1 to 20 hours, about 2 hours, for about 4 hours or for about 18 hours.

26. The method of claim 25, wherein said enzymatic digestion conditions are held for about 4 hours.

27. The method of claim 17, wherein an amount of said peptide digest injected on LC-MS ranges from about 1 μg to 2 μg.

28. A method for identifying, quantifying, and/or characterizing at least one non-viral protein in a sample including at least one virus or viral vector, comprising:
(a) subjecting a sample including at least one non-viral protein and at least one virus or viral vector to partially denaturing conditions to form a partially denatured sample, wherein said partially denaturing conditions are capable of substantially denaturing said at least one non-viral protein and do not substantially denature said at least one virus or viral vector;
(b) subjecting said partially denatured sample to digestion conditions to form a peptide digest; and
(c) subjecting said peptide digest to LC-MS analysis to identify, quantify, and/or characterize said at least one non-viral protein.

29. The method of claim 28, wherein said at least one non-viral protein is a host cell protein.

30. The method of claim 28, wherein said at least one virus or viral vector is an AAV vector.

31. The method of claim 30, wherein said AAV vector comprises a serotype selected from a group consisting of AAV1, AAV2, AAV3, AAV4, AAV5, AAV6, AAV7, AAV8, AAVrh8, AAV2/8, AAV9, AAV10, AAV11, AAV12, variations thereof and combinations thereof.

32. The method of claim 28, wherein said viral vector is a therapeutic vector.

33. The method of claim 28, further comprising subjecting said sample to a reducing step prior to or concurrent with step (a).

34. The method of claim 33, wherein said reducing step comprises contacting said sample to TCEP or DTT.

35. The method of claim 28, wherein said partially denaturing conditions comprise a temperature from about 35° C. to about 65° C.

36. The method of claim 35, wherein said temperature is about 35° C., about 40° C., about 45° C., about 50° C., about 55° C., about 65° C., about 70° C. or about 75° C.

37. The method of claim 28, wherein said digestion conditions comprise contacting said partially denatured sample to at least one digestive enzyme, optionally wherein said at least one digestive enzyme comprises trypsin.

38. The method of claim 37, wherein said trypsin is present at an enzyme to substrate ratio of from 1:10 to 1:4000, about 1:20, about 1:400, or about 1:2000.

39. The method of claim 28, wherein a duration of said digestion conditions is from 1 to 20 hours, about 2 hours, for about 4 hours or for about 18 hours.

40. The method of claim 28, wherein an amount of said peptide digest injected on LC-MS is from about 1 μg to about 4 μg, about 1 μg, about 1.5 μg, about 2 μg, about 3 μg, or about 4 μg.

41. The method of claim 28, further comprising quantifying a ratio of the abundance of said at least one non-viral protein to an abundance of a protein of said at least one virus or viral vector.

42. The method of claim 41, wherein said ratio is at least 0.5, from about 0.5 to 10, about 0.5, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10.

43. The method of claim 29, wherein said host cell protein is a Chinese hamster ovary (CHO) protein or a human protein.

44. The method of claim 28, wherein said liquid chromatography system is selected from a group consisting of reversed phase chromatography, ion exchange chromatography, size exclusion chromatography, affinity chromatography, hydrophobic interaction chromatography, hydrophilic interaction chromatography, mixed-mode chromatography, or a combination thereof.

45. The method of claim 28, wherein said mass spectrometry system is electrospray ionization mass spectrometer, nano-electrospray ionization mass spectrometer, or an Orbitrap-based mass spectrometer, wherein said mass spectrometer is coupled to said liquid chromatography system.

46. A method for characterizing at least one HCP in a sample including a virus or viral vector, comprising:
(a) quantifying and/or characterizing said HCP outside of said virus or viral vector according to the method of claim 28;
(b) quantifying and/or characterizing said HCP inside of said virus or viral vector according to the method of claim 11; and
(c) comparing the quantification and/or characterization of step (a) to the quantification and/or characterization of step (b) to characterize said at least one HCP.

47. A method for enriching a virus or viral vector, comprising:
(a) subjecting a sample including a virus or viral vector and at least one non-viral protein to partially denaturing conditions to produce a partially denatured sample, wherein said partially denatured conditions are capable of substantially denaturing said at least one non-viral protein and do not substantially denature said virus or viral vector;
(b) subjecting said partially denatured sample to enzymatic digestion conditions to form a partially digested sample, wherein said enzymatic digestion conditions are capable of substantially digesting denatured proteins and do not substantially denature native proteins; and
(c) subjecting said partially digested sample to a separation step to enrich said virus or viral vector.

48. The method of claim 47, wherein said at least one non-viral protein is a host cell protein.

49. The method of claim 47, wherein said at least one virus or viral vector is an AAV vector.

50. The method of claim 49, wherein said AAV vector comprises a serotype selected from a group consisting of AAV1, AAV2, AAV3, AAV4, AAV5, AAV6, AAV7, AAV8, AAVrh8, AAV2/8, AAV9, AAV10, AAV11, AAV12, variations thereof and combinations thereof.

51. The method of claim 47, wherein said viral vector is a therapeutic vector.

52. The method of claim 47, further comprising subjecting said sample to a reducing step prior to or concurrent with step (a).

53. The method of claim 52, wherein said reducing step comprises contacting said sample to TCEP or DTT.

54. The method of claim 47, wherein said partially denaturing conditions comprise a temperature from about 35° C. to about 65° C.

55. The method of claim 54, wherein said temperature is about 35° C., about 40° C., about 45° C., about 50° C., about 55° C., about 65° C., about 70° C. or about 75° C.

56. The method of claim 47, wherein said digestion conditions comprise contacting said partially denatured sample to at least one digestive enzyme, optionally wherein said at least one digestive enzyme comprises trypsin.

57. The method of claim 56, wherein said trypsin is present at an enzyme to substrate ratio of from 1:10 to 1:4000, about 1:20, about 1:400, or about 1:2000.

58. The method of claim 47, wherein a duration of said digestion conditions is from 1 to 20 hours, about 2 hours, for about 4 hours or for about 18 hours.

59. The method of claim 48, wherein said host cell protein is a Chinese hamster ovary (CHO) protein or a human protein.

60. The method of claim 47, wherein said separation step comprises centrifugation.

61. A method for enriching at least one fragment of at least one non-viral protein in a sample including at least one virus or viral vector, comprising:
(a) subjecting a sample including at least one non-viral protein and at least one virus or viral vector to partially denaturing conditions to produce a partially denatured sample;
(b) subjecting said partially denatured sample to digestion conditions to produce a peptide digest; and
(c) subjecting said peptide digest to a separation step to enrich said at least one fragment of said at least one non-viral protein.

62. The method of claim 61, wherein said at least one non-viral protein is a host cell protein.

63. The method of claim 61, wherein said at least one virus or viral vector is an AAV vector.

64. The method of claim 63, wherein said AAV vector comprises a serotype selected from a group consisting of AAV1, AAV2, AAV3, AAV4, AAV5, AAV6, AAV7, AAV8, AAVrh8, AAV2/8, AAV9, AAV10, AAV11, AAV12, variations thereof and combinations thereof.

65. The method of claim 61, wherein said viral vector is a therapeutic vector.

66. The method of claim 61, further comprising subjecting said sample to a reducing step prior to or concurrent with step (a).

67. The method of claim 66, wherein said reducing step comprises contacting said sample to TCEP or DTT.

68. The method of claim 61, wherein said partially denaturing conditions comprise a temperature from about 35° C. to about 65° C.

69. The method of claim 68, wherein said temperature is about 35° C., about 40° C., about 45° C., about 50° C., about 55° C., about 65° C., about 70° C. or about 75° C.

70. The method of claim 61, wherein said digestion conditions comprise contacting said partially denatured sample to at least one digestive enzyme, optionally wherein said at least one digestive enzyme comprises trypsin.

71. The method of claim 70, wherein said trypsin is present at an enzyme to substrate ratio of from 1:10 to 1:4000, about 1:20, about 1:400, or about 1:2000.

72. The method of claim 61, wherein a duration of said digestion conditions is from 1 to 20 hours, about 2 hours, for about 4 hours or for about 18 hours.

73. The method of claim 62, wherein said host cell protein is a Chinese hamster ovary (CHO) protein or a human protein.

74. The method of claim 61, wherein said separation step comprises centrifugation.
